SEQUENCE LISTING (1 sequence)

SEQ ID NO: 1 - protein, 8 aa, Gallus gallus
SIINFEKL

(19) United States
(12) Patent Application Publication
(10) Pub. No.: US 2024/0067956 A1
(43) Pub. Date: Feb. 29, 2024

(54) METHODS AND COMPOSITIONS FOR MOLECULAR TRACKING IN INDIVIDUAL CELLS IN VIVO

(71) Applicant: The Regents of the University of Colorado, a body corporate, Denver, CO (US)

(72) Inventors: Beth A. Jiron Tamburini, Denver, CO (US); Jay R. Hesselberth, Denver, CO (US); Shannon Marie Walsh, East Lansing, MI (US)

(21) Appl. No.: 18/271,727

(22) PCT Filed: Jan. 13, 2022
(86) PCT No.: PCT/US22/12235

§ 371 Date: Jul. 11, 2023

Related U.S. Application Data
(60) Provisional application No. 63/136,798, filed on Jan. 13, 2021.

(51) Int. Cl. C12N 15/10; C12Q 1/6804; C12Q 1/6841

(52) U.S. Cl. CPC C12N 15/1065; C12Q 1/6804; C12Q 1/6841

(57) ABSTRACT

The present invention relates to a molecular tracking device to follow distribution, acquisition, and retention of antigens in cells, such as in the lymphatic system of a subject. Molecular conjugates including an antigen conjugated to a nuclease-resistant tag are tracked using single-cell mRNA sequencing. The molecular tracking devices allow new approaches to investigate dynamic tissue dissemination of antigens and identify new mechanisms of antigen acquisition and retention at cellular resolution in vivo.

### CROSS-REFERENCE TO RELATED APPLICATION

This application claims priority to U.S. Provisional Application Ser. No. 63/136,798, filed Jan. 13, 2021, the disclosure of which is incorporated herein by reference in its entirety.

### STATEMENT REGARDING FEDERALLY SPONSORED RESEARCH

This invention was made with Government Support under Federal Grant Nos. RO1 AI121209, T32 A1007405, R35 GM119550, T32 A1074491, and R21 AI155929, awarded by the National Institutes of Health (NIH). The U.S. Government has certain rights in this invention.

### STATEMENT REGARDING ELECTRONIC FILING OF A SEQUENCE LISTING

A Sequence Listing in ASCII text format, entitled 151077-00044_ST25.txt, 555 bytes in size, generated on Jan. 12, 2022, and filed via EFS-Web, is provided with this application. The Sequence Listing is incorporated herein by reference into the specification for its disclosures.

### FIELD OF THE INVENTION

The present invention relates to methods and compositions related to tracking uptake and distribution of molecules and/or molecular structures, such as antigens and antigenic molecules and/or molecular structures, resulting from, for example, an infection or vaccination.

### BACKGROUND

Depending on the route of infection, vaccination mode, and ability of antigens to traffic, different dendritic cell (DC) subsets are required to initiate T cell priming. Upon subcutaneous immunization, small soluble proteins and virus particles pass through the lymphatics to the lymph node (LN), where LN-resident DCs acquire and present antigen^{1,2}. For larger antigens and/or pathogens that are too large to pass through the lymphatic capillaries, dermal DCs migrate to the LN for presentation of processed antigens to naïve T cells^{1,2,3,4}. Most adaptive immune responses require antigen processing and presentation by conventional dendritic cells in either the draining lymph node or at the site of infection or vaccination (migratory cutaneous or dermal DCs)⁵.

Previous studies have shown that viral antigens persist in the lymph node beyond the time frame of infectious virus^{6,7,8,9,10,11}. Lymphatic endothelial cells (LEC) were recently found to store antigens from viral infection and vaccination^{12,13,14}. Using a vaccine formulation that elicits robust cell-mediated immunity comprised of antigen, a Toll-like receptor (TLR) agonist, and an agonistic aCD40 antibody (TLR/αCD40 vaccination) or viral infection^{15,16,17,18,19,20,21,22,23,24,25,26}, it was discovered that antigens were durably retained in the lymph node^{12,13,14}. Antigen storage was dependent on the presence of a TLR agonist (e.g. polyI:C alone (TLR3/MDA5/RIGI or Pam3cys (TLR1/2)+αCD40), but also occurred with antigen conjugated to a TLR agonist (e.g. 3M019 (TLR7))¹⁴. This process has been described as “antigen archiving,” and it has been shown that this process is important to poise memory T cells for future antigenic encounters¹⁴.

Prior to these studies, the only non-hematopoietic cell type thought to retain antigens were follicular dendritic cells, which harbor antigens in antigen-antibody complexes for extended periods of time and for the benefit of B cell memory^{11,27}. Fibroblasts and non-endothelial stromal cells comprise a large portion of the lymph node stroma and are capable of presenting peripheral tissue antigens, but their capacity to acquire and present foreign antigens is not yet well understood^{28,29,30}. Initial studies were unable to detect antigen archiving by blood endothelial cells or fibroblasts^{12,13}. While LECs have been shown to present antigens in the absence of inflammation to induce T cell tolerance^{31,32,33,34,35,36,37,38}, it has been shown that presentation of archived antigen occurs only after exchange of the archived antigen from an LEC to a migratory DC; changing the stimulus from tolerizing to immunostimulatory^{12,13}. Soluble antigens are exchanged via two distinct mechanisms: (i) direct exchange between LECs and migratory DCs and (ii) LEC death. Antigen transfer from LECs to both migratory conventional (c)DC1s and cDC2s is required for archived antigen presentation to antigen-specific memory T cells^{12,13}. After viral infection, archived antigen is transferred to Batf3 dependent migratory DCs as a result of LEC death during lymph node contraction¹².

Limitations of current approaches have precluded sensitive and quantitative measures of antigen levels across cell types, providing only a glimpse of the cell types and molecular mechanisms that control antigen acquisition, processing and retention in the lymph node. Studies of antigen in the lymph node and peripheral tissues have mainly relied on antigen-fluorophore conjugates or indirect measurement of antigen uptake and presentation^{2,6,7,11,12,14,39}, which defined antigen acquisition by specific DC subsets and trafficking of antigens using live imaging². However, antigen archiving has been difficult to study because antigen-fluorophore conjugates suffer from low microscopic detection sensitivity, yielding weak signals that diminish over time. Moreover, detection of antigen in the lymph node and other tissues has relied on flow cytometric analysis using cell surface markers, restricting analysis to specific cell types. Furthermore, shortcomings related to the use of antigen/antibody-DNA conjugates in tracking antigens include the rapid degradation of these conjugates in vivo. Thus, in order to address these limitations in current approaches to detecting antigen acquisition and/or uptake, improved approaches to molecular tracking, such as in tracking antigen molecules throughout tissue-specific cell types in vivo, are needed.

### SUMMARY

The present inventive concept relates to methods and compositions of molecular tracking in individual cells in vivo using single-cell mRNA-sequencing. According to an aspect of the inventive concept, provided is a molecular conjugate including: a molecule or molecular structure of interest; and a nucleic acid linked to the molecule or molecular structure of interest, wherein the nucleic acid includes a nucleic acid amplification primer binding sequence, an identifier sequence, and a capture sequence, and wherein the nucleic acid includes a phophorothioate modification at every nucleotide linkage.

According to another aspect of the inventive concept, provided is a method of tracking acquisition of a molecule or molecular structure of interest in cells including: exposing a subject to a molecular conjugate of the inventive concept; performing single-cell nucleic acid sequencing on cells of interest derived and/or isolated from the subject; and analyzing data acquired from the single-cell nucleic acid sequencing for a presence of the identifier sequence, wherein the presence of the barcode identifier sequence is indicative of acquisition of the molecule or molecular structure of interest by a particular cell or cells.

According to another aspect of the inventive concept, provided is a method of tracking distribution or location of a molecule or a molecular structure of interest in a subject including: exposing a subject to the molecular conjugate of the inventive concept; performing single-cell nucleic acid sequencing on cells of interest derived and/or isolated from the subject; and analyzing data acquired from the single-cell nucleic acid sequencing for a presence of the barcode identifier sequence, wherein the presence of the barcode identifier sequence is indicative of acquisition of the molecule or molecular structure of interest by a particular cell or cells in the subject, and where the molecule or molecular structure of interest is distributed or located in the subject.

According to another aspect off the inventive concept, provided is a method of molecular tracking including: exposing a subject to the molecular conjugate of the inventive concept; performing single-cell nucleic acid sequencing on cells of interest derived and/or isolated from the subject; and analyzing data acquired from the single-cell nucleic acid sequencing for a presence of the barcode identifier sequence, wherein the presence of the barcode identifier sequence is indicative of acquisition of the molecule of interest by a particular cell or cells.

According to another aspect of the inventive concept, provided is a method of tracking antigen archiving including: exposing a subject to a molecular conjugate including an antigen of interest linked to a nucleic acid, wherein the nucleic acid includes a nucleic acid amplification primer binding sequence, a barcode identifier sequence, and a capture sequence, and wherein the nucleic acid includes a phophorothioate modification at every nucleotide linkage; performing single-cell nucleic acid sequencing on cells of interest derived and/or isolated from the subject; and analyzing data acquired from the single-cell nucleic acid sequencing for a presence of the barcode identifier sequence, wherein the presence of the barcode identifier sequence is indicative of acquisition of the antigen of interest by a particular cell or cells.

### BRIEF DECRIPTION OF THE DRAWINGS

FIG. 1. Antigen-psDNA conjugates undergo normal processing and presentation. Panel a. Schematic of ovalbumin (PDB code 1ova) antigen conjugation to barcoded DNA with phosphodiester and phosphorothioate DNA linkages and a biotin label (circle with B inside). Sulfur replaces a non-bridging oxygen to create a DNA phosphorothioate linkage. List of oligo sequences used can be found in Supplementary Table 1 from Walsh et al. doi.org/10.1101/2020.08.19.219527v1 (2020). Panel b. Conjugation of oligonucleotides to ovalbumin. Purified conjugate was analyzed by 10% TBE native PAGE stained with GelRed for DNA (left) followed by Coomassie staining for protein (right). DNA-TCO=61nt barcoded oligonucleotide with 5′-trans-cyclooctene (TCO); ova-mTZ=ovalbumin functionalized with methyltetrazine (mTZ); ova-DNA=DNA-conjugated ovalbumin product with oligonucleotide attached. Panel c. Bone marrow derived dendritic cells (BMDC) were treated with pDNA, psDNA, ova-pDNA or ova-psDNA (5 μg) by addition to the culture media. After 1, 3, and 7 days cells were washed, released, lysed and analyzed for pDNA or psDNA by qPCR. Values are displayed as fold-change relative to the negative control (cells alone). Asterisks denote sample significant amounts relative to the negative control (P<0.01; Wilcoxon rank-sum test). Panel d. Flow cytometric analysis of ova-psDNA conjugates acquired by BMDCs after 1 day or 7 days. Cells were washed 1 day after ova-psDNA treatment. Harvested BMDCs were stained with anti-ovalbumin made in rabbit and a secondary anti-rabbit conjugated to Phycoerythrin (PE) and then stained with streptavidin conjugated to brilliant violet 421 to visualize the 3′ biotin label on the psDNA. Shown are average and ±standard error. Experiment was performed three times with three technical replicates. Panel e. As in (panel d) except cells were plated onto glass coverslips and treated with ova-psDNA for 24 hr prior to staining with either anti-ovalbumin and a secondary conjugated PE (lower left) followed by streptavidin conjugated to Fluorescene Isothiocyanate (FITC) (lower right). Co-localization is shown in yellow. Scale bar is 10 μm. Imaging was repeated three independent times Approximately 100 cells were visualized with a similar frequency of double-positive cells observed in (panel d) No single-positive cells were detected. Panel f. Analysis of DNAs as in (panel c) using murine lymph node lymphatic endothelial cells. Panel g. BMDCs were incubated with ova-psDNA (conjugated), ova plus psDNA (unconjugated), or PBS for 1, 3, 7 days prior to adding OT-1 T cells labeled with violet proliferation dye. T cells and BMDCs were co-cultured at a ratio of 1:10 for 3 days. Panel h. Quantification of results shown in (panel g) using the percent divided calculation described in the methods. Experiments were performed three times with 3-5 wells per sample with similar results. Error bars represent SEM. Asterisks denote sample significant amounts relative to the negative control (P<0.05 Wilcoxon rank-sum test). Exact p-values are: Day 1 psDNA:ova-psDNA p=0.008, psDNA:untreated p=0.016, ova-psDNA:untreated p=0.016; Day 3 psDNA:ova-psDNA p=0.008, psDNA:untreated p=0.016, ova-psDNA:untreated p=0.016; Day 7 psDNA:ova-psDNA p=1, psDNA:untreated p=0.400, ova-psDNA:untreated p=0.400. n.d.: none detected.

FIG. 2. Antigen-psDNA conjugates elicit a robust immune response in vivo. Panel a. Mice were immunized in the footpad with ova alone or ova-psDNA with or without polyI:C/aCD40 or polyI:C. After seven days, draining popliteal LNs were harvested and cells were stained and gated as B220−, CD3+, CD8+, CD44+ and OVA257 Kb OVA257-264-specific tetramer to measure antigen specific CD8 T cell responses. Panel b. Quantification of OVA257-264-specific CD8 T cells within the lymph node (data from results shown in panel a). Panel c. As in (panel a) and (panel b) except cells were restimulated with SIINFEKL (SEQ ID NO:1, OVA257-264) peptide for 6 hrs ex vivo in the presence of brefeldin A, then stained for IFNg and IL-2. Panel d. Quantitation of interferon gamma positive CD8+ T cells with or without peptide stimulation in the draining lymph node. Experiment was performed 3 times; shown is combined data from at least 3 mice per group, per experiment. P-values were calculated using a two stage step up method of Benjamin, Krieger and Yekutieli and did not assume consistent standard deviation. Error bars represent SEM.

FIG. 3. Dynamic acquisition of antigen-psDNA conjugates in lymph node tissue. (Panels a, d, g, j) UMAP projections are shown for DCs (panels a, d), LECs (panel g), and FRCs (panel j) at day 2 (panel a) and day 14 (panels d, g, j). (Panels b, e, h, k) Relative ova signal was calculated by dividing antigen counts for each cell by the median antigen counts for T and B cells. Signals are plotted on log10 scale; black dots indicate median values and vertical lines denote quartiles. Statistical comparisons between each pair of groups is available in Supplementary Table 2 from Walsh et al. doi.org/10.1101/2020.08.19.219527v1 (2020). (Panels c, f, i, 1) UMI-adjusted antigen counts are displayed on UMAP projections for each cell type.

FIG. 4. Antigen-based classification of DCs and validation of genes associated with DC activation. (Panels a, e) Day 2 (panel a) and day 14 (panel e) cDC2 Tbet− cells containing low and high antigen counts were identified using a two component mixture model. A UMAP projection is shown for ova-low and ova-high cells. Cell types not included in the comparison are shown in white (other). (Panels b, f) The distribution of ova antigen counts is shown for ova-low and ova-high cDC2 Tbet− cells. Dotted lines indicate the mean counts for each population. (Panels c, g) UMAP projections show the expression (log-normalized counts) of top markers associated with ova-high cDC2 Tbet− cells. (Panels d, h) Expression (log-normalized counts) of antigen-high markers in each cell type.

FIG. 5. Antigen-based classification of LECs and identification of marker genes. Panel a. Day 14 LECs were classified into antigen-high and antigen-low using a two-component Gaussian mixture model. A UMAP projection is shown for antigen-low and antigen-high cells. T cells, B cells, and epithelial cells are shown in white (Other). Panel b. Distribution of antigen counts for antigen-low (light) and antigen-high (dark) cells. Dotted lines indicate mean counts for each population. Identification of genes associated with ovalbumin (ova)-low and ova-high for each cell type is available in Supplementary Table 3 from Walsh et al. doi.org/10.1101/2020.08.19.219527v1 (2020). Panel c. The fraction of cells belonging to each LEC cell type for antigen-low and antigen-high populations. Panel d. UMAP projections show expression of genes significantly enriched in the antigen-high population (scale is log-normalized counts). Panel e. Expression (log-normalized counts) of antigen-high markers in each cell type. Panel f. Mice were injected in the footpad with nystatin (dose) and 1 hour later OVA488/polyI:C/αCD40. After 24 hours, mice were euthanized and draining popliteal LN removed, stained for LEC markers (CD45, PDPN, CD31, PDL1) and gated as in FIG. 13. Shown are representative flow flots and quantification of geometric mean fluorescence intensity (gMFI) from naïve (black bar), vehicle control (red bar) and nystatin treated (blue bar). Three mice per group were evaluated and experiment was performed 3 independent times with similar results. Nystatin treatment reduces OVA488 signal in LECs relative to vehicle (P=0.02; Wilcoxon rank-sum test).

FIG. 6: Visualization of antigen and DNA in different cell types. Panel a. Gating and single-color staining controls for anti-ovalbumin (ova) and streptavidin 1 day post addition of ova-psDNA. Panel b. Flow cytometry of SV-40 transformed endothelial cells (SVEC) with indicated stains at indicated time points as in (a). Experiment was repeated three times with three replicates. Panel c. Immunofluorescence of SVECs treated with ova-psDNA for 24 hr using anti-ova followed by a secondary anti-rabbit conjugated to PE and streptavidin conjugated with FITC. Cells were mounted with Vectashield with DAPI. Scale bar is 10 μm. Approximately 100 cells were visualized with a similar frequency of double-positive cells as detected in (b). No single-positive cells were detected. Panel d. Gating strategy for murine lymphatic endothelial cells. Panel e. Amount of ovalbumin conjugated to Alexa Fluor 488 that was acquired over a 2-7 day period in the presence or absence of type 1 IFN. Panel f. Quantification of fluorescence intensity two or seven days after treatment as indicated. Experiment was performed at least three times with three replicates. g. Flow cytometry of bone marrow- derived macrophages (BMDMs) with indicated stains at indicated time points as in (a). Experiment was repeated three times with three replicates.

FIG. 7: DNA barcode is not retained in cell media over time. a. Bone marrow derived dendritic cells (BMDC) were grown for 7 days and cultured in GM-CSF. After 7 days 5 mg of either pDNA, psDNA, ova-pDNA or ova-psDNA were added to the culture media and 1, 3, or 7 days after addition, media was removed, and qPCR was performed on using primers against the DNA added. Panel b. As in panel a except with murine lymph node lymphatic endothelial cells. Panel c. As in FIG. 1, panel h, BMDCs were treated with ovalbumin (ova) (5 μg)±polyI:C and anti-CD40 (20 m each) or ova-psDNA±polyI:C and anti-CD40 overnight and then co-cultured with Carboxyfluorescein Succinimidyl Ester (CFSE)-labeled OT1 T cells for 3 days before evaluating CFSE dilution by flow cytometry. Experiments were performed three times with 3-5 wells per sample with similar results.

FIG. 8. Gating strategies. Panel a. Gating strategy for tetramer staining and intracellular cytokine staining of CD8 T cells. Panel b. Gating strategy used for cell sorting prior to single cell RNA sequencing.

FIG. 9. Vaccinia plus ova-DNA conjugate induces ova specific T cell response and archiving. Panel a. Mice were vaccinated in the footpad with 10 μg of ovalbumin-pDNA (phosphodiester backbone) or ovalbumin-psDNA (phosphorothioate backbone) with or without vaccinia virus (10³ PFU). Each barcode conjugate contained a unique sequence barcode. Seven days after vaccination, mice were euthanized and draining popliteal LN cells were isolated and stained. Shown are B220−, CD3+, CD8+ cells. Box indicates the frequency of tetramer specific and CD44 high cells per lymph node. Panel b. Quantification of results shown in panel a. Panel c. Mice were vaccinated with 10 μg ovalbumin conjugated to phosphorothioate DNA (ova-psDNA) plus 1E3 VV-WR, 1E4 VV-WR or polyI:C/αCD40 (5 μg each) in each footpad. LNs were harvested twenty days later and Miltenyi bead selection was performed using CD45 to select hematopoietic (CD45+) versus non-hematopoietic (CD45−) cells. The amount of DNA barcode was assessed in each group as a faithful reporter of antigen archiving. Values are displayed as fold-change relative to the negative control naïve sample. Experiment was performed twice with 3-4 mice per group.

FIG. 10. Detection of DNA barcode requires conjugation to ovalbumin. Mice were vaccinated with ova-psDNA, psDNA and pDNA with IE3 CFU of VV-WR. Each DNA injected had a unique barcode sequence for detection during sequencing. Counts of ova-psDNA, psDNA, pDNA for DCs at (Panel a). day 2 or (Panel b). day 14. Counts for ova-psDNA, psDNA and pDNA for (Panel c). LECs and (Panel d). FRCs 14 days post vaccination.

FIG. 11. Antigen counts were independent of total mRNA counts. Antigen counts were compared with total mRNA counts for each cell for DCs (panels a, b), LECs (panels c, d) and FRCs (panels e, f). Pearson's correlation coefficient and associated p-value is shown for each cell type.

FIG. 12. LEC cell types associated with high antigen counts 2 days after vaccination. Panel a. A UMAP projection is shown for LEC cell types, epithelial cells, B cells and T cells identified for the day 2 timepoint. Panel b. Relative ova signal is shown for each cell type. Relative ova signal was calculated by dividing antigen counts for each cell by the median antigen counts for T and B cells. Panel c. Antigen counts are displayed on the UMAP projection shown in A. Panels d, e. Correlation coefficients are shown comparing each identified LEC cell type with the reference cell types from Xiang et al.

FIG. 13: Antigen is held by PD-LVICAM1 High LECs. Panel a. Gating strategy for LECs in the dLN with markers for ICAM1 and PD-L1. Panel b. Representative flow plots of antigen held by LECs 14 days following subcutaneous immunization with OVA488 (10 mg/site), polyI:C (5 mg/site) and aCD40 (5 mg/site). LECs were gated first on PD-L1 high/ICAM1 high or PD-L1 low/ICAM1 low and then gated on antigen positive. Panel c. Quantification results shown in of panel b showing the number of antigen positive LECs gated on PD-L1 high/ICAM1 high or PD-L1low/ICAM1 low. Statistical analysis was done using an unpaired student's t test, ** p=0.0032

FIG. 14. FRC cell types with high antigen counts at day 2 post vaccination. Panel a. UMAP projection is shown for FRC cell types, B cells, and T cells identified for the day 2 timepoint. Panel b. Relative ovalbumin signal is shown for each cell type. Relative ovalbumin signal was calculated by dividing antigen counts for each cell by the median antigen counts for T and B cells. Panel c. Antigen counts are displayed on the UMAP projection shown in Panel a.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Unless otherwise defined, all technical terms used herein have the same meaning as commonly understood by one of ordinary skill in the art to which this disclosure belongs. The definitions contained in this specification are provided for clarity in describing the components, compositions, and methods herein and are not intended to limit the claimed aspects and embodiments of the inventive concept.

### Definitions

Articles “a,” “an,” and “the” are used herein to refer to one or to more than one (i.e., at least one) of the grammatical object of the article. By way of example, “an element” means at least one element and can include more than one element. The term “and/or” includes any and all combinations of one, or more, of the associated listed items and may be abbreviated as “/”.

The term “comprise,” as used herein, in addition to its regular meaning, may also include, and, in some embodiments, may specifically refer to the expressions “consist essentially of” and/or “consist of.” Thus, the expression “comprise” can also refer to, in some embodiments, the specifically listed elements of that which is claimed and does not include further elements, as well as embodiments in which the specifically listed elements of that which is claimed may and/or does encompass further elements, or embodiments in which the specifically listed elements of that which is claimed may encompass further elements that do not materially affect the basic and novel characteristic(s) of that which is claimed. For example, that which is claimed, such as a method, kit, system, etc. “comprising” listed elements also encompasses, for example, a composition, method, kit, etc. “consisting of,” i.e., wherein that which is claimed does not include further elements, and a composition, method, kit, etc. “consisting essentially of,” i.e., wherein that which is claimed may include further elements that do not materially affect the basic and novel characteristic(s) of that which is claimed.

The term “molecular conjugate” refers to a construct including a molecule and/or molecular structure of interest linked (covalently, non-covalently, or otherwise as described herein) to a nucleic acid. the nucleic acid may include several functional elements, such as: a binding sequence for a nucleic acid amplification primer, also referred to, in some embodiments, as a PCR handle; an identifier sequence, for example, a barcode identifier sequence that specifically identifies that linked/attached molecule and/or molecular structure of interest; and a capture sequence that includes a sequence that is complementary to, for example, a binding/hybridizing sequence included on a gel bead, such as is available from 10× Genomics, but not limited thereto. The nucleic acid may further include a unique molecular identifier (UMI) locate, for example, either 3′ or 5′ to the identifier/barcode identifier sequence.

A single strand of a nucleic acid typically includes a 5′ (5-prime) end and a 3′ (3-prime) end. The terms 5′ and 3′ therefore refer to a relative position on a single strand of a nucleic acid. Accordingly, the relative position of certain elements or sequences of a nucleic acid (e.g., a nucleic acid amplification primer binding sequence/amplification or PCR handle, an identifier sequence/barcode and a capture sequence) can be specified in a sequential order from 5′ to 3′, or alternatively from 3′ to 5′. It will be appreciated that generally, elements/sequences of a nucleic acid are referred to from 5′ to 3′, unless otherwise specified. For example, a nucleic acid may include, from 5′ to 3′, a nucleic acid amplification primer binding sequence, an identifier sequence/barcode and a capture sequence, and may be represented as: 5′-PCR handle-barcode-capture sequence-3′. In the above example, the barcode and the capture sequence may be referred to as being 3′ of the PCR handle. Also, in the above example, the handle and the barcode may be referred to as being 5′ of the capture sequence. Further, the position of the handle in the above example may also be referred to as adjacent to the barcode. Similarly, the barcode may be referred to as flanked by the handle and the anchor. Accordingly, one of skill in the art would know what is meant by the positional terms 3′ and 5′. Such positional language, as used herein, unless explicitly indicated otherwise, does not imply that additional nucleic acid sequences are not interposed between the reference elements. For example, in the above example, additional sequences (e.g., a UMI) may be present between the PCR handle and the barcode, or the barcode and the capture sequence.

The terms “linked to” or “attached to” as used herein to describe the interaction between the components of the conjugates include covalent linkages/attachments or a variety of non-covalent types of linkages/attachments. Chemistries useful in assembling the constructs described herein include, but are not limited to, thiol-maleimide, thiol-haloacetate, amine-NHS, amine-isothiocyanate, and “click” chemistries, such as, but not limited to, Copper(I)-catalyzed azide-alkyne cycloadditions (CuAAC), strain-promoted azide-alkyne cycloadditions (SPAAC) and inverse electron-demand Diels-Alder (IEDDA) additions, such as a methyltetrazine-trans-cyclooctene addition. In some embodiments, each nucleic acid is linked to the molecule or molecular structure of interest by an irreversible covalent link. In other embodiments, each nucleic acid is linked to the molecule or molecular structure of interest by a cleavable covalent link, for example a disulfide link or a photocleavable linker.

A “molecule of interest” may be any naturally occurring or synthetic biological or chemical molecule. In some embodiments, the molecule of interest may be a peptide or protein. In some embodiments, the molecule of interest may be any antigen or antigenic molecule. A “molecular structure of interest” may refer to a feature and/or characteristic of a molecule of interest. For example, in some embodiments, the molecular structure may include an amino acid or nucleic acid sequence, or the molecular structure may include a sugar and/or glycan chain. In some embodiments, the molecular structure may include a primary sequence, or a secondary or tertiary structure of a peptide, protein, or nucleic acid, or a secondary of tertiary structure of a sugar/glycan chain. In some embodiments, the molecule/molecular structure of interest may be an active agent, such as a component of a therapeutic/pharmaceutical composition/formulation, such as a vaccine.

In general, the nucleic acid/oligonucleotide can be any length that accommodates the lengths of its functional components. In some embodiments, the nucleic acid/oligonucleotide is between about 20 and 100 nucleic acid bases/nucleotides in length. In some embodiments, the nucleic acid/oligonucleotide sequence is at least about 20, 30, 40, 50, 60, 70, 80, 90 or over 100 nucleic acid bases/nucleotides in length. In other embodiments, the nucleic acid/oligonucleotide is about 200 to about 400 monomeric components, e.g., nucleic acid bases/nucleotides in length. In some embodiments, the nucleic acid is generally made up of deoxyribonucleic acids (DNA). In some embodiments, the nucleic acid is a DNA sequence. In some embodiments, the nucleic acid, or a part/portion thereof, includes modified DNA bases. Modification of DNA bases are known in the art and can include chemically modified bases including labels. In other embodiments, the nucleic acid, or a part/portion thereof, includes ribonucleic acid (RNA) sequences or modified ribonucleotide bases. Modification of RNA bases are known in the art and can include chemically modified bases including labels. In still other embodiments, different portions of nucleic acid/oligonucleotide sequence can include DNA and RNA, modified bases/nucleobases, or modified nucleic acid base/nucleotide connections, such as, for example, phosphorothioate DNA, peptide nucleic acids (PNAs) and locked nucleic acids (LNAs). In some embodiments, the nucleic acid/oligonucleotide includes phosphorothioate modifications, for example, such as phosphorothioate DNA, at some, most, or all nucleic acid base/nucleotide connections/linkages of the nucleic acid/oligonucleotide.

The term “amplification handle,” “amplification sequence” or, in some embodiments, “PCR handle” refers to a functional component of the nucleic acid/oligonucleotide sequence which itself is an oligonucleotide or polynucleotide sequence that provides a binding/annealing/hybridizing site and/or sequence for amplification of the nucleic acid/oligonucleotide sequence by, for example, a nucleic acid amplification primer. The amplification or PCR handle can be formed of DNA, RNA, phosphorothioate DNA, PNA, LNA, modified bases or combinations of these bases, or polyamides, etc. In some embodiments, the amplification or PCR handle is about 10 nucleic acid bases/nucleotides in length. In other embodiments, the amplification or PCR handle is at least about 5 to 100 nucleic acid bases/nucleotides in length. Thus in various embodiments, the amplification or PCR handle is formed of a sequence of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 nucleic acid bases/nucleotides. In some embodiments, when present in multiple nucleic acid/oligonucleotide sequences, the amplification or PCR handle can be the same or different, depending upon the techniques intended to be used for amplification. In some embodiments, the amplification or PCR handle can be a generic sequence suitable as an annealing site for a variety of amplification technologies. Amplification technologies include, but are not limited to, DNA-polymerase based amplification systems, such as polymerase chain reaction (PCR), real-time PCR, loop mediated isothermal amplification (LAMP, MALBAC), strand displacement amplification (SDA), multiple displacement amplification (MDA), recombinase polymerase amplification (RPA) and polymerization by any number of DNA polymerases (for example, T4 DNA polymerase, Sulfulobus DNA polymerase, Klenow DNA polymerase, Bst polymerase, Phi29 polymerase) and RNA-polymerase based amplification systems (such as T7-, T3-, and SP6-RNA-polymerase amplification), nucleic acid sequence based amplification (NASBA), self-sustained sequence replication (3SR), rolling circle amplification (RCA), ligase chain reaction (LCR), helicase dependant amplification (HDA), ramification amplification method and RNA-seq.

The term “barcode” describes a nucleic acid/oligonucleotide sequence, that when it is a functional element of the nucleic acid, is specific for a single molecular conjugate. The barcode can be formed of a defined sequence of DNA, RNA, modified bases or combinations of these bases, as defined above. In some embodiments, the barcode is about 2 to 4 monomeric nucleic acid bases/nucleotides in length. In other embodiments, the barcode is at least about 1 to about 100 nucleic acid bases/nucleotides in length. Thus in various embodiments, the barcode is formed of a sequence of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 nucleic acid bases/nucleotides in length. According to embodiments of the inventive concept, the barcode may be used in identifying uptake of a molecular conjugate or conjugates by a particular cell or cells.

The term “capture sequence” refers to a polynucleotide or oligonucleotide sequence, which is designed to hybridize to a complementary oligonucleotide sequence, e.g., an oligonucleotide, a primer, and/or the like. In some embodiments of the nucleic acid, a capture sequence is designed for the purpose of generating a double-stranded oligonucleotide sequence. In some embodiments, the capture sequence is positioned at the 3′ end of the nucleic acid. In other embodiments, the capture sequence is positioned at the 5′ end of the nucleic acid. In some embodiments, each capture sequence is specific for its intended complementary sequence. For example, in certain embodiments, the capture is configured to hybridize to a 3′ end of a complementary oligonucleotide such that the 3′ end of the complementary oligonucleotide acts as a primer that can generate a second complementary strand of the oligonucleotide in the presence of a polymerase. When used in the various methods described herein, a capture sequence may hybridize to a free complementary sequence or with a complementary sequence that is immobilized on a substrate. In some embodiments, the capture sequence can be formed of a sequence of, e.g., DNA, RNA, phosphorothioate DNA, PNA, LNA, modified bases or combinations of these bases, or polyamides, etc. In some embodiments, the capture sequence is about 3 to 15 nucleic acid bases/nucleotides in length. In other embodiments, the capture sequence can be at least about 3 to 100 nucleic acid bases/nucleotides in length. In some embodiments, an anchor comprises 3 to 100, 3 to 50, 3 to 30, 5 to 30, 10 to 20, 5 to 20, or 5 to 15 nucleic acid bases/nucleotides in length. In various embodiments, an Anchor is formed of a sequence of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 nucleic acid bases/nucleotides in length.

A “complementary oligonucleotide, “complementary binding oligonucleotide,” “complementary hybridizing oligonucleotide,’ “complementary oligo,” “complementary binding oligo” or “complementary hybridizing oligo” may be, e.g., an oligonucleotide/polynucleotide including at least a sequence that is complementary to the capture sequence of the molecular conjugate. In some embodiments, the complementary oligo is not part of the molecular conjugate; rather it is any oligonucleotide/polynucleotide that is part of a construct-purification kit or an mRNA-sequencing kit. The term “complementary sequence” may refer to a sequence to which a capture sequence (or other nucleic acid, e.g., a primer or capture oligonucleotide) is intended to hybridize/bind to, often resulting in a hybridized double stranded complex. In the presence of a polymerase, a hybridized complex can, in some embodiments, be extended in a 3′ direction where a nucleic acid template is present. Accordingly, in certain embodiments, a sequence complementary to the capture sequence can hybridize to a capture sequence thereby providing a primer for amplification and/or to generate a double stranded sequence. In some embodiments, the complementary oligonucleotide/polynucleotide may include sequences that can be used as amplification/PCR handles and optionally include one or more UMIs and barcode sequences. In the methods of the inventive concept, the extension of the complementary oligonucleotide/polynucleotide, with its complementary sequence hybridized to the capture sequence copies the barcode, the UMI and the amplification/PCR handle from the molecular conjugate onto the capture polymer/oligonucleotide. According to embodiments of the inventive concept, the capture polymer/oligonucleotide and its complementary sequence can be formed of DNA, RNA, phosphorothioate DNA, PNA, LNA, modified bases or combinations of these bases, or of any other component as defined above. Depending upon the assay steps involved and the intended target, the complementary sequence can be unhindered or “free” in the biological sample. In one embodiment, the complementary oligonucleotide/polynucleotide includes a sequence that is a primer sequence designed to participate in amplifying the molecular conjugate oligonucleotide sequence. In other embodiments, the complementary oligonucleotide/polynucleotide is immobilized on a substrate. Similar to the capture sequence, each complementary sequence can be at least about 3 to about 100 nucleic acid bases/nucleotides in length. Thus in various embodiments, the complementary sequence is formed of a sequence of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 nucleic acid bases/nucleotides in length. according to embodiments of the inventive concept, the complementary sequence may be any oligonucleotide sequence that may be envisioned by one of skill in the art, provided that it can hybridize to its intended capture sequence.

The term “immobilized” can refer to an oligonucleotide/polynucleotide including, for example, a complementary sequence as described herein, that is attached to a solid substrate resulting in reduction or loss of mobility through, for example, physical adsorption through charge-charge interaction or hydrophobic interaction, covalent bonding, Streptavidin-Biotin interaction, or affinity coupling.

The term “substrate” can refer to a microparticle/bead, a microfluidics microparticle/bead, a slide, a multi-well plate or a chip. The substrates are conventional and can be glass, plastic or of any conventional materials suitable for the particular assay or diagnostic protocols. In exemplary embodiments, substrates include gel beads, for example, gel beads-in-emulsion (GEMs) such as those that are commercially available from 10× Genomics, and the like.

### Compositions

The compositions and formulations used in methods of the inventive concept as described herein include molecular conjugates that include components selected based on the purpose of the assay/method, and the protocols used for the assay/method. The method used may dictate the selection and compositions of the various components described above which make up the composition. It will be appreciated that the following description is not exhaustive, and one of skill in the art can design many different compositions based on the teachings provided herein. The composition may also include the molecular conjugates in a suitable carrier or excipient. The elements of each composition will depend upon the assay that may be employed.

In some embodiments, a molecular conjugate may include a molecule or molecular structure of interest attached to or conjugated with a nucleic acid by, for example, a linker. The nucleic acid may be an oligonucleotide sequence including: a hybridizing/binding sequence for a nucleic acid amplification primer, i.e., an amplification handle and/or PCR handle; an identifier sequence, such as a barcode, that specifically identifies the molecule or molecular structure of interest; and a capture sequence for hybridizing/binding to a complementary oligonucleotide sequence to the capture sequence. The nucleic acid may, optionally, further include a UMI, which may by positioned adjacent to the identifier sequence/barcode, at either the 5′ or 3′ end of the identifier sequence/barcode.

The nature of the molecule or molecular structure of interest is not particularly limited, and as such may be any molecule or molecular structure that one of skill in the art may wish to track/monitor uptake of the molecule/molecular structure by a cell or cells that may be individual cells or part of a higher organism. Such tracking/monitoring may be tracking/monitoring of uptake of the molecule/molecular structure in a cell, cells, organs, and/or tissues of a subject. In some embodiments, for example, the molecule or molecular structure of interest may be one that behaves as an antigen or may have antigenic properties, such as a toxin or foreign substance/organism, which triggers an immune and/or inflammatory response in a subject. In some embodiments, the molecule or molecular structure of interest may be, for example, a therapeutic for promoting an immune response against a pathogen. In some embodiments, the molecule or molecular structure of interest may be a polypeptide/protein or a sugar/glycan/peptidoglycan, or a structure on a polypeptide/protein or structure on a sugar/glycan/peptidoglycan. In some embodiments, the molecule or molecular structure may be, for example, a polypeptide and/or protein of bacterial origin or nature. In some embodiments, the molecule or molecular structure may be a polypeptide and/or protein of viral origin or nature, for example, a polypeptide and/or protein from an influenza virus, or a polypeptide and/or protein from a coronavirus, such as SARS coronavirus-2 (SARS-CoV2). In other embodiments, the molecule or molecular structure of interest is nonimmunogenic, i.e., does not trigger an immune and/or inflammatory response against the molecule or molecular structure of interest, such as a therapeutic for treating a disorder, such as a viral infection. In some embodiments, the molecule or molecular structure is an active agent/part of an active agent that is a component in and/or an ingredient included in a vaccine.

Many types of molecules or molecular structures of interest, amplification primer binding sequences/PCR handles, and identifier sequences/barcodes can be used to generate a wide variety of molecular conjugates/compositions as described herein without departing from the scope of the inventive concept.

Kits containing the molecular conjugates/compositions of the inventive concept are also provided. Such kits will contain one or more molecular conjugates/compositions, one or more preservatives, stabilizers, or buffers, and such suitable assay and amplification reagents depending upon the amplification and analysis methods and protocols with which the molecular conjugate/composition will be used. Still other components in a kit include optional reagents for cleavage of the linker, a wash buffer, a blocking solution, a lysis buffer, and an encapsulation solution, detectable labels, immobilization substrates, optional substrates for enzymatic labels, as well as other laboratory items and instructions for use.

### Methods of Use

The molecular conjugates/compositions, and kits including the molecular conjugates/compositions of the inventive concept described herein may be used in a variety of methods by employing any number of assays/methods for detecting the molecular conjugate/composition in general.

In some embodiments, methods of the inventive concept include methods of tracking a molecule or molecular structure of interest, i.e., a method of molecular tracking, in a subject. In some embodiments, methods of tracking include tracking acquisition and/or uptake of the molecule or molecular structure of interest in the subject. In some embodiments, methods of tracking include tracking distribution or location of the molecule or molecular structure of interest in the subject. In some embodiments, methods of tracking include methods of tracking antigen archiving in the subject.

Methods of tracking, according to embodiments of the inventive concept, may include use of molecular conjugates/compositions as described herein to determine the presence, amount, or absence of the molecular conjugate in a sample. In some embodiments, the presence, amount, or absence of the molecular conjugate in a sample is a measure of the presence, amount, or absence of the molecular conjugate in a single cell or cells. According to some embodiments, methods of the inventive concept include methods of molecular tracking, for example, but not limited to, tracking molecular acquisition/uptake, such as tracking and quantifying antigen uptake, distribution, and/or archiving in a subject and tracking and quantifying antigen uptake, distribution, and/or archiving within cells and/or tissues of the subject. In some embodiments, the cells and/or tissues of the subject in which methods of the inventive concept may be performed include any particular cell or cells of interest in which single-cell nucleic acid sequencing may be performed. In some embodiments, the particular cell or cells of interest may be CD45+ cells. In some embodiments, the particular cell or cells of interest may be CD45− cells. In some embodiments, cells and/or tissues of the subject in which molecular tracking can be performed, for example, tracking antigen distribution and/or archiving, include the lymphatic system, for example, including primary and secondary lymphoid organs and tissues. In some embodiments, molecular tracking, molecular acquisition, antigen uptake, distribution, and/or archiving within cells and/or tissues of the subject are the sites at which lymphocyte activation by antigens takes place, such as the lymph nodes, spleen, and/or Peyer's patches, and cells therein, such as endothelial cells, epithelial cells, and dendritic cells, such as those found in stromal cells, such as lymph node stromal cells, as well as monocytes, T cells and/or B cells. Lymph node stromal cells in which molecular tracking/molecular acquisition/antigen uptake/antigen archiving may be followed/monitored include: fibroblastic reticular cells (FRCs); marginal reticular cells (MRCs); follicular dendritic cells (FDCs); lymphatic endothelial cells (LECs); blood endothelial cells (BECs); alpha-7 integrin pericytes (AlPs); and double negative cells (DNCs). In some embodiments, the particular cell or cells in which molecular tracking/molecular acquisition/antigen uptake/antigen archiving may be followed/monitored include FDCs and/or LECs. Exemplary surface markers expressed by lymph node stromal cells include glycoprotein CD31 and glycoprotein podoplanin GP38. Different sub-populations of lymph node stromal cells are also known by their production of small molecules; where they are located; and their function. Most also express common markers such as desmin, laminin, various subunits of integrins, vascular cell adhesion molecule 1 (VCAM-1) and mucosal vascular addressin cell adhesion molecule 1 (MAdCAM-1). LECs in which molecular tracking/molecular acquisition/antigen uptake/antigen archiving may be followed/monitored include, for example, Ptx3 LECs, ceiling LECs, Marco LECs, floor LECs, and/or subcapsular LECs.

Methods of tracking the presence, amount, or absence, and/or tracking change in the presence, amount, or absence of the molecular conjugate in a sample, according to embodiments of the inventive concept, may include methods of single-cell nucleic acid sequencing. The methods of nucleic acid sequencing, such as methods of single-cell nucleic acid sequencing, that may be used in the methods of the inventive concept are not particularly limited, and any suitable nucleic acid sequencing method that would be appreciated by one of skill in the art can be used to sequence the nucleic acids described herein and/or to detect the presence, amount or absence of the various nucleic acids, molecular conjugates, oligonucleotides, amplification products and barcodes described herein. Exemplary methods of single-cell sequencing used in the methods according to the inventive concept include methods of single-cell RNA, in particular mRNA, sequencing. Methods of single-cell mRNA sequencing and methods of analysis of sequencing data/results used in the methods according to the inventive concept include, for example, methods as described in Walsh et al. bioRxiv preprint doi.org/10.1101/2020.08.19.219527v1 (2020), Walsh et al. eLife 10:e62781 doi.org/10.7554/eLife.62781 (2021), and U.S. Patent Application Publication No. 2018/0251825, the disclosures of each of which are incorporated herein by reference.

It will be appreciated that methods of molecular tracking of the inventive concept include tracking of molecules of interest for presence and/or distribution, and changes in presence and/or distribution thereof, within a cell, cells, and/or tissues derived from a subject, such as derived, extracted and/or isolated cells, such as cells derived/extracted/isolated from an organ from the subject, and/or cells derived/extracted/isolated from tissues or tissue samples/tissue slices from the subject, may be performed at multiple/a plurality of time points and/or over a period of time following exposure of the cell, the cells and/or the tissues in a subject and/or derived from a subject to a molecular conjugate including the molecule of interest. The period of time following exposure is not particularly limited, and may be for example, over about 1, 2, 3, 4, 5, 6, 7 days (1 week), 8, 9, 10, 11, 12, 13, or 14 days (2 weeks) after exposure. In some embodiments, the period of time may be about 1, 2, 3, or 4 weeks after exposure. In some embodiments, the period of time may be about 1, 2, 3, 4, 5, 6, 7, 8, 9, 10, 11, or 12 months (1 year) after exposure. In some embodiments, the period of time may be about 1-3 months, about 1-6 months, or about 1-12 months, or even over longer periods of time (years) after exposure.

Subjects suitable in which the composition, compositions and formulations of the present inventive concept may be used, and/or subjects from which samples, such as a cell, cells, tissues, and/or tissue samples for analysis may be derived include, but are not limited to, mammalian subjects. Mammals according to the present inventive concept include, but are not limited to, canines, felines, bovines, caprines, equines, ovines, porcines, rodents (e.g., rats and mice), lagomorphs, primates, humans and the like, and mammals in utero. Any mammalian subject in need of using methods according to the present inventive concept is suitable. In some embodiments of the present inventive concept, the subject is a human subject. The human subject treated according to methods of the present inventive concept may be of any gender (for example, male, female or transgender) and at any stage of development (i.e., neonate, infant, juvenile, adolescent, adult, elderly).

The nature/manner in which the molecular conjugates, compositions, and/or formulations of the inventive concept may be administered/exposed to a cell, cells, and/or tissues in a subject or derived from a subject is not particularly limited, and any suitable route of administration/exposure of the molecular conjugates, compositions, and/or formulations of the inventive concept (e.g., parenteral, enteral, oral, nasal, inhalational, ocular, transmucosal and/or transdermal, etc.) to a subject, or exposure of the molecular conjugates, compositions, and/or formulations of the inventive concept to a cell, cells and/or tissues in a subject or derived from a subject may be used as would be appreciated by one of skill in the art. Nonetheless, in terms of administration, in some embodiments, the most suitable route may depend on the nature and manner that a subject may be exposed to the antigen/antigenic agent of interest and tracked by the molecular conjugates, compositions, and/or formulations of the inventive concept. In some embodiments, routes of administration are parenteral, including, but not limited to, for example, intravenous, subcutaneous, intramuscular, intradermal, intramucosal, intraperitoneal, etc. administration/injection. In some embodiments, the parenteral administration may by intravenous or intraperitoneal administration. In some embodiments, routes of administration are oral, including, but not limited to, for example, buccal, enteral, sublabial, sublingual, etc. administration. In some embodiments, the routes of administration may be transmucosal administration. In some embodiments, the routes of administration are nasal or intranasal administration. Such administration may be inhalational or direct application to a mucosal membrane.

Having described various aspects of the present inventive concept, the same will be explained in further detail in the following examples, which are included herein for illustration purposes only, and which are not intended to be limiting to the inventive concept.

### EXAMPLES

### Example 1: Materials and Methods

Key resources used in these examples are listed in the following table:

Key resources table

| Reagent type | | | | |
|---|---|---|---|---|
| (species) or | | Source or | | Additional |
| resource | Designation | reference | Identifiers | information |
| Antibody | Anti-mouse CD40 | BioXcell | Cat#BE0016-2 | |
| | (Rat monoclonal) | | | |
| Antibody | Anti-ovalbumin | Abcam | Ab181688 | 1:100 |
| | (rabbit | | | |
| | monoclonal) | | | |
| Antibody | Anti-rabbit IgG PE | Biolegend | Cat# 406421 | 1:100 |
| | (Donkey | | RRID: AB_2563484 | |
| | polyclonal) | | | |
| Antibody | Anti-mouse CD45 | Biolegend | Cat#103138 | 1:300 |
| | BV510 (Rat | | RRID: AB_2563061 | |
| | monoclonal) | | | |
| Antibody | Anti-mouse CD45 | Biolegend | Cat#103106 | 1:300 |
| | PE (Rat | | RRID: AB_312971 | |
| | monoclonal) | | | |
| Antibody | Anti-mouse | Biolegend | Cat#127410 | 1:200 |
| | podoplanin APC | | RRID: AB_10613649 | |
| | (Hamster | | | |
| | monoclonal) | | | |
| Antibody | Anti-mouse CD31 | Biolegend | Cat#102420 | 1:200 |
| | PerCP-Cy5.5 (Rat | | RRID: AB_10613644 | |
| | monoclonal) | | | |
| Antibody | Anti-mouse PD-L1 | Biolegend | Cat#124315 | 1:200 |
| | BV421 (Rat | | RRID: AB_10897097 | |
| | monoclonal) | | | |
| Antibody | Anti-mouse CD8a | Biolegend | Cat#100714 | 1:400 |
| | APC-Cy7 (Rat | | RRID: AB_312753 | |
| | monoclonal) | | | |
| Antibody | Anti-mouse CD44 | Biolegend | Cat# 103032 | 1:400 |
| | PerCP-Cy5.5 (Rat | | RRID: AB_2076204 | |
| | monoclonal) | | | |
| Antibody | Anti-mouse | Biolegend | Cat# 103248 | 1:300 |
| | B220/CD45R | | RRID: AB_2650679 | |
| | BV510 (Rat | | | |
| | monoclonal) | | | |
| Antibody | Anti-mouse | Biolegend | Cat# 103208 | 1:300 |
| | B220/CD45R PE | | RRID: AB_312993 | |
| | (Rat monoclonal) | | | |
| Antibody | Anti-mouse | Biolegend | Cat#117324 | 1:400 |
| | CD11c APC Cy7 | | RRID: AB_830649 | |
| | (Hamster | | | |
| | monoclonal) | | | |
| Antibody | Anti-mouse | Biolegend | Cat#101216 | 1:300 |
| | CD11b PE-Cy7 | | | |
| | (Rat monoclonal) | | | |
| Peptide, | Streptavidin | Biolegend | Cat#405226 | 1:1000 |
| recombinant | BV421 | | | |
| protein | | | | |
| Peptide, | Streptavidin | Thermo Fisher | Cat#S11223 | 1:1000 |
| recombinant | AF488 | Scientific | | |
| protein | | | | |
| Chemical | PolyI:C | Invivogen | Cat#Vac-PIC | |
| compound, | | | | |
| drug | | | | |
| Chemical | Nystatin | Sigma Aldrich | Cat#N4014 | |
| compound, | | | | |
| drug | | | | |
| Chemical | Violet | BD Biosciences | Cat#562158 | |
| compound, | proliferation dye | | | |
| drug | | | | |
| Chemical | CFSE | BD Biosciences | Cat#565082 | |
| compound, | | | | |
| drug | | | | |
| Strain, strain | WT: C57BL/6 | Charles River | C57BL/6 (B6) Mouse | |
| background | | Labs | Inbred 027 | |
| (Mus | | | | |
| musculus) | | | | |
| Strain, strain | OT1: C57BL/6-Tg | Jackson Labs | JAX: 003831 | |
| background | (TcraTcrb) | | | |
| (M. | 1100Mjb/J | | | |
| musculus) | | | | |
| Cell line (M. | SVEC4-10 | ATCC | ATCC CRL2181 | |
| musculus) | | | | |

Mice. 4-6 week old mice were purchased from Charles River or Jackson Laboratory, unless otherwise stated, bred and housed in the University of Colorado Anschutz Medical Campus Animal Barrier Facility. Wild type and OT1 mice were all bred on a C57BL/6 background. OT1 mice are a TCR transgenic strain specific to the SIINFEKL (SEQ ID NO:1) peptide of ovalbumin (OVA257-264) in the context of H-2K^{b}. All animal procedures were approved by the Institutional Animal Care and Use Committee at the University of Colorado.

Phosphorothioate and phosphodiester oligonucleotides. Oligonucleotides were synthesized by Integrated DNA Technologies (IDT) and contained a 5′ amine for conjugation, primer binding site, barcode, 10×Genomics Gel Bead Primer binding site for capture sequence 2, and a 3′ biotin. Phosphorothioated oligonucleotides contained a phosphorothioate modification at every linkage. All oligonucleotide sequences can be found in Supplementary Table 1 from Walsh et al. doi.org/10.1101/2020.08.19.219527v1 (2020), the disclosure of which is incorporated herein by reference. Supplementary Table 1 from Walsh et al. lists antigen tags and other oligonucleotide sequences used in qPCR and single-cell experiments

Conjugation of oligonucleotides to protein. Oligonucleotides were conjugated to ovalbumin by iEDDA-click chemistry⁸⁹. Oligonucleotides were derivatized with trans-cyclooctene (TCO) in 10× borate buffered saline (BBS; 0.5 M borate, 1.5 M NaCl, pH 7.6; sterile filtered). Dilution of this buffer to 1× results in a final pH of 8.5. A reaction containing 40 nmol of amine-modified oligo (0.5 mM), 1× BBS, 10% DMSO, 8 μL of 100 mM TCO-PEG4-NHS in DMSO (10 mM final; Click Chemistry Tools, A137), pH 8.5 was rotated at room temperature for 15 min. A second aliquot containing the same amount of TCO-PEG4-NHS in DMSO was added and the reaction was rotated at room temperature for another hour. Excess NHS was quenched by adding glycine, pH 8.5 to a final concentration of 20 mM and rotated at room temperature for 5 min. Modification was confirmed by analysis on an 8% denaturing TBE PAGE gel. Samples were precipitated by splitting the reaction into 20 μL aliquots and adding 280 μL of nuclease-free water, 30 μL of 3 M NaCl, and 990 μL of 100% ethanol. The precipitation reaction was incubated at −80° C. overnight followed by centrifugation at >10,000×g for 30 min. The supernatant was discarded, the pellet was washed with 100 μL of 75% ethanol, and centrifuged at >10,000×g for 10 min. The supernatant was removed, and the pellets were dried for 5 min at room temperature. The pellets were recombined by resuspension in 50 μL of 1× BBS. Samples were quantified by A260.

To conjugate methyltetrazine to ovalbumin: detoxified Ovalbumin (Sigma-Aldrich, St. Louis, MO) (using a Triton X-114 lipopolysaccharide detoxification method⁹⁰), was buffer exchanged into 1× BBS, pH 8.5. To an Amicon 0.5 mL 30 kDa filter (Millipore, UFC5030) was added 1 mg of ovalbumin and 1× BBS to a volume of 450 μL. The filter was centrifuged at 14,000×g for 5 min. The flow through was discarded and the sample washed twice with 400 of 1× BBS. The product-containing column was inverted into a clean collection tube and centrifuged at 1,000×g for 2 min. Assuming no loss, the volume of the sample was adjusted to 2 mg/mL with 1× BBS. 400 μL of 1× BBS was added to the Amicon filter and stored at 4° C. for later use. A 500 μL labeling reaction containing 0.5 mg of ovalbumin in 1× BBS and 50 μL of 2 mM mTz-PEG4-NHS in DMSO (0.2 mM final; Click Chemistry Tools, 1069), pH 8.5 was rotated at 4° C. overnight. Excess NHS was quenched by adding glycine, pH 8.5 to a final concentration of 20 mM and rotated at room temperature for 10 min. The previously stored Amicon filter was centrifuged at 14,000×g for 5 min and the flow through discarded. 400 μL of reaction mixture was added to the filter and centrifuged at 14,000×g for 5 min. This was repeated until all 1 mg of protein had been added to the filter and was supplemented with 1× BBS as needed. Samples were washed 1× with 400 μL of 1× BBS. The product-containing column was inverted into a clean collection tube and centrifuged at 1,000×g for 2 min. Assuming no loss, the volume of the sample was adjusted to 5 mg/mL with 1× BBS.

For the final antigen-DNA conjugation, a 100 μL reaction containing 300 μg of ovalbumin-mTz and 6 nmol of oligonucleotide-TCO (1:1 equivalents) in 1× BBS was rotated at 4° C. overnight. Excess mTz was quenched with 10 μL of 10 mM TCO-PEG4-glycine and rotated at room temperature for 10 min. TCO-PEG4-glycine was prepared by reaction of 10 mM TCO-PEG4-NHS with 20 mM glycine, pH 8.5 in 1× BBS for 1 h at room temperature and stored at −20° C. Products were analyzed by 10% TBE PAGE. For purification, excess ovalbumin and DNA were removed by filter centrifugation. 200 μL of 1× PBS was added to an Amicon 0.5 mL 50 kDa filter (Millipore, UFC5050) followed by 300 μL of sample. The filter was centrifuged at 14,000×g for 5 min and the flow through discarded. Samples were washed five times with 400 of 1× PBS and centrifuged at 14,000×g for 5 min. The product-containing column was inverted into a clean collection tube and centrifuged at 1,000×g for 2 min. Purified products were analyzed by 10% TBE PAGE and total protein quantified with Bio-Rad protein quantification reagent (Bio-Rad, 5000006). LPS contamination after conjugation was below 0.5 EU/mg as mentioned below in the vaccinations section.

Bone marrow derived dendritic cell and lymphatic endothelial cell cultures. Both left and right tibia and femur were isolated under sterile conditions. Bone marrow was extracted from femurs of 6-8-week-old C57BL/6 mice by decollating the top and bottom of the bone and releasing the marrow with 27 gauge syringe and 5 ml of Modified Essential Medium (MEM) (Cellgro). Suspension was strained through 100 μm filter, pressed with the back of a syringe and washed. Cells were spun 1500 RPM, 5 min then suspended in MEM with 10% FBS, 20 ng/ml of GM-CSF from the supernatant of the B78hi-GM-CSF cell line. Every 2 days, dead cellular debris was spun, supernatant collected and combined 1:1 with new 40ng/ml GM-CSF 20% FBS (2×) in MEM. After 7 days of culturing at 37C, 5% CO₂, cells were harvested for respective assays. Mouse lymphatic endothelial cells (Cell Biologics, C57-6092) were cultured in Endothelial Cell Media (Cell Biologics, M1168) with kit supplement. T75 Flasks were coated with gelatin for 30 minutes 37° C., washed with PBS and then inoculated with mLEC. Cells were passaged with passive trypsin and split at a density of 1:3. For BMDMs, whole bone marrow was isolated and red blood cells were lysed. Cells were then cultured in M-CSF (50 ng/mL) for 6 days in complete media. Cells were harvested via cell scraper and plated for treatment.

Conjugate detection assay. BMDC and mLEC Cultures were stimulated with 20 μg of anti-CD40, 20 μg Poly I:C, and 5 μg of either OVA-psDNA or OVA in a 6-well format. 24 hr post treatment, cells were washed and refreshed with new media. At designated time points, cells were harvested, counted, and transferred into micro-centrifuge tubes, spun at 350 g, and both supernatant and pellets were frozen at −80° C. Cell pellets were lysed in 50 μL of Mammalian Protein Extraction Reagent (MPER; Thermo Scientific, 78503). Conjugate DNA was measured by qPCR amplification from 1 μL of lysate in a 10 μL reaction containing 5 μL of iTaq Universal SYBR Green Supermix (Bio-Rad, 1725125) and 5 pmol of each primer (Supplementary Table 1 from Walsh et al.). Quantification was measured using an external standard curve and normalized to lysate protein content. To visualize within ova-psDNA acquisition by cells, cells were fixed with 10% formalin for 10 min at room temperature in the dark, washed with PBS, and spun for 10 min at 2000 rpm. Cells were then permeabilized with 100% ice-cold methanol for 20 min at −20° C. Cells were then washed with PBS and spun as above. Cells were stained with the anti-ova antibody as above for at least 2 hr at room temperature and then washed with 1% bovine serum albumin (BSA) with sodium azide (FACS buffer) and spun as above. Cells were then incubated with an anti-rabbit secondary in PE for 1 hr at room temperature and then washed with FACS buffer. Cells were then stained with streptavidin conjugated to BV421 in PBS for 15 min at room temperature and then washed twice with FACS buffer prior to acquiring cells on a FACS CANTO II flow cytometer. Analysis was performed using FlowJo software. Immunofluorescence was performed as above except cells were grown on glass coverslips and stained on cover slips using an anti-rabbit dylight 649 and streptavidin-FITC. Coverslips were mounted with Vectashield with DAPI and imaged on a Zeiss LSM780 confocal microscope. The imaging experiments were performed in the Advanced Light Microscopy Core part of the NeuroTechnology Center at University of Colorado Anschutz Medical Campus

OT1 Isolation and co-culture. CD8 T cells were isolated from an OT1+ mouse using the mojosort mouse CD8 T cell isolation kit (Biolegend) and labeled with violet proliferation dye (BD Biosciences cat #562158). For DC-T cell co-culture, BMDCs were treated with psOVA (5 μg), or OVA+psDNA (5 μg) for 1,3 or 7 days. BMDCs were washed and then co-cultured with labeled OT1s for three days at a 1:10 ratio of BMDC:OT1. Cells were then stained and run on a flow cytometer. OT1 division (percent dividing cells) was calculated as previously described⁹¹ using the equation fraction

$$diluted = {\sum }_{1}^{i} \frac{{N}_{i}}{{2}^{i}} / {\sum }_{0}^{i} \frac{{N}_{i}}{{2}^{i}}$$

where i is the generation number (0 is the undivided population), and Nᵢ is the number of events in generation i.

Vaccinations. 6-8 week-old C57BL/6 (CD45.2) mice were immunized with 1E3 or 1E4 colony forming units of Vaccinia Western Reserve or 5 μg of Poly I:C (Invivogen) with or without 5 μg of anti-CD40 (FGK4.5, BioXcell) and 10 μg of OVA-psDNA or OVA in 50 μl volume by footpad injection. Endotoxin levels were quantified using the Pierce Limulus Amebocyte Lysate Chromogenic Endotoxin Quantitiation kit (ThermoScientific) to be less than 0.5 EU/mg for either ovalbumin or ovalbumin conjugated to psDNA.

Nystatin. Nystatin (Sigma N4014) was resuspended in DMSO to a concentration of 10 mg/mL Mice were injected with 50 μL of 10 mg/mL nystatin per footpad one hour prior to injection with ovalbumin conjugated to Alexa 488 (5 μg) in a mixture with polyI:C and anti-CD40 (2.5 μg each). LNs were harvested and digested as below (preparation of single cell suspensions) and stained with CD45 brilliant violet 510 (Biolegend clone 30F11, 1:300), PDPN APC (Biolegend clone 8.1.1, 1:200), CD31 PercP Cy5.5 (Biolegend clone 390, 1:200) and PD-L1 pacific blue (Biolegend clone 10F.9G2, 1:200).

Tetramer and intracellular cytokine assays. Draining LNs were processed by glass slide maceration 7 days after injection, washed and suspended in FACS (2% FBS in PBS) buffer containing Tetramer (OVA257-264)-PE (1:400) (NIH tetramer core facilty), CD8 APC-Cy7 (Biolegend clone 53-6.7 1:400) for 1 hr at 37° C. Cells were washed and stained for 30 minutes in CD44 PerCP Cy5.5 (Biolegend clone IM7, 1:400), B220 BV510 (Biolegend clone RA3-6B2, 1:300). Samples were run on the FACs Canto II flow cytometer (BD).

Preparation of single-cell suspensions. Two days or two weeks following vaccination with 1E3 CFU of VV-WR with 10 μg is of ova-psDNA per foot pad, popliteal LNs were removed from 15 mice and LNs were pulled-apart with 22-gauge needles. Tissue was digested with 0.25 mg of Liberase DL (Roche, Indianapolis, IN) per ml of EHAA media with DNAse (Worthington, Lakewood, NJ) at 37 degrees. Every 15 minutes media was removed, cells spun down and new digestion media added to the undigested tissue until no tissue remained, ˜1 hr. Following digestion cells were filtered through a screen and washed with 5 mM EDTA in EHAA. LN cells were then divided into thirds where one third underwent staining with CD11c (N418), CD11b and B220 and a live/dead dye (Tonbo). Live cells were then sorted into 4 tubes on a FACs Aria Cell Sorter (BD): sorted CD11c-APC Cy7 (Biolengend clone N418 1:400)+ cells, sorted CD1lb PE-Cy7 (Biolegend clone M1/70)+ cells, sorted B220 PE (Biolegend clone RA3-6B2)+ cells and Fixable Viability Stain 510 (BD Biosciences Cat #546406) ungated live cells which were recombined at a 4:4:1:1 ratio, respectively. For the remaining two-thirds of cells, cells were stained with CD45 PE followed by magnetic bead isolation using the Miltenyi bead isolation kit. CD45 negative cells that passed through the column were then washed. Both sorted and selected (CD45+ and CD45=) cells were then washed with PBS in 0.1% BSA as described in the Cell Prep Guide (10× Genomics) and counted using a hemacytometer. Final concentration of cells was approximately 1000 cells/ul and approximately 10-20 μL were assayed.

Single-cell library preparation using the 10× Genomics platform. Cells were assayed using the 10× Genomics single-cell 3′ expression kit v3 according to the manufacturer's instructions (CG000183 Rev B) and CITE-seq protocol (cite-seq.com/protocol Cite-seq_190213) with the following changes:
(1) cDNA amplification and cleanup. During cDNA amplification, 1 μL of 0.2 μM each mixture of additive forward and reverse primers (Supplementary Table 1 from Walsh et al.) was included to amplify the antigen tags. The CITE-seq protocol was followed for size selection and cleanup of the cDNA and antigen tag products. Antigen tag products were eluted in 60 μL of nuclease-free water.
(2) Amplification of antigen tag sequencing libraries. A 100 μL PCR reaction was prepared containing 45 μL of purified antigen tag products, 1× Phusion HF Buffer (NEB), 200 μM dNTPs, 25 pmol each Illumina sequencing forward and reverse primers (Supplementary Table 1 from Walsh et al.), 2 Units Phusion High Fidelity DNA Polymerase. PCR cycling conditions were 95° C. for 3 min, 6-10× (95° C. for 20 s, 60° C. for 30 s, 72° C. for 20 s), 72° C. for 5 min. Products were purified according to the CITE-seq protocol. Gene expression and antigen tag libraries were analyzed on the Agilent D1000 Tapestation and quantified using the Qubit HS dsDNA fluorometric quantitation kit (Thermo Scientific).
   All libraries were sequenced on an Illumina NovaSeq 6000 with 2×150 base pair read lengths.

Transcriptome and oligonucleotide detection and analysis. Briefly, FASTQ files from the gene expression and antigen tracking libraries were processed using the feature barcode version of the cellranger count pipeline (v3.1.0). Reads were aligned to the mm10 and Vaccinia virus (NC_006998) reference genomes. Analysis of gene expression and antigen tracking data was performed using the Seurat R package (v3.2). Antigen tracking and gene expression data were combined into the same Seurat object for each sample (CD45−/day 2, CD45+/day 2, CD45−/day 14, CD45+/day 14). Cells were filtered based on the number of detected genes (>250 and <5000) and the percent of mitochondrial reads (<15%). Gene expression counts were log normalized (NormalizeData), and relative ova signal was calculated by dividing ova-psDNA counts by the median ova-psDNA counts for all T and B cells present in the sample. To allow for the values to be log-transformed for visualization, a pseudo count was added (smallest non-zero value*0.5).

Gene expression data were scaled and centered (ScaleData). 2000 variable features (Find VariableFeatures) were used for PCA (RunPCA) and the first 40 principal components were used to find clusters (FindNeighbors, FindClusters) and calculate uniform manifold approximation and projection (UMAP) (RunUMAP). Cell types were annotated using the R package clustifyr (rnabioco.github.io/clustifyr)⁴⁸ along with reference bulk RNA-seq data from ImmGen (available for download through the clustifyrdata R package, rnabioco.githublo/clustifyrdata). To annotate cell subtypes, the samples were then divided into separate objects for DCs, LECs, and FRCs. Cell subsets were then annotated using clustifyr with reference bulk RNA-seq data for DCs^{49,50}, FRCs⁵¹, and LECs^{52,53,54}. After assigning DC, LEC, and FRC subtypes, the other cell types (TB cells, epithelial cells, NK cells) were added back to the objects and reprocessed as described above.

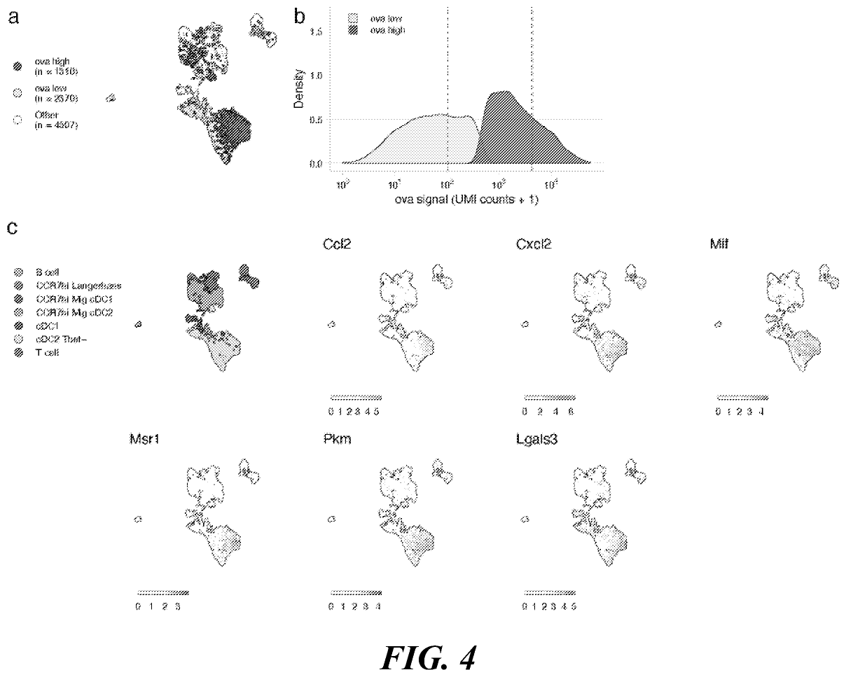

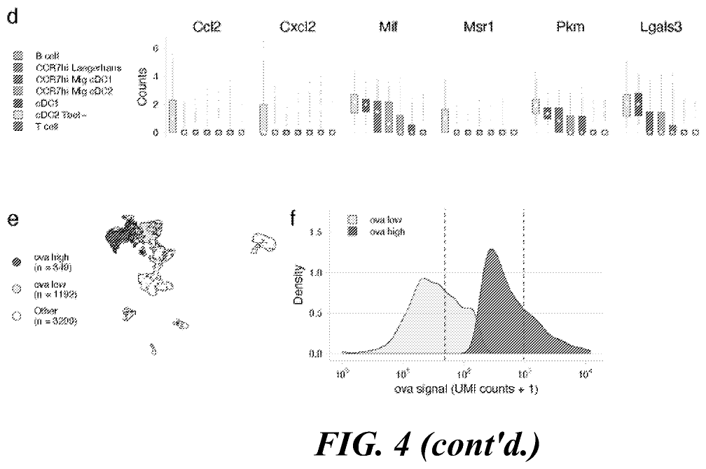

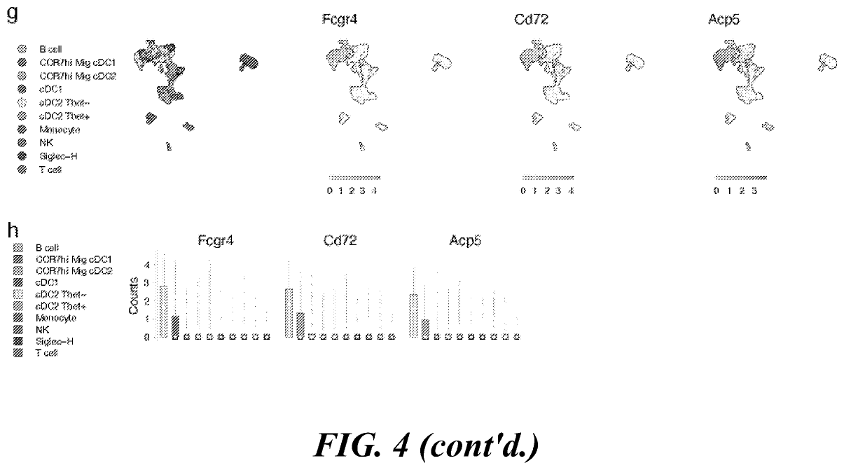

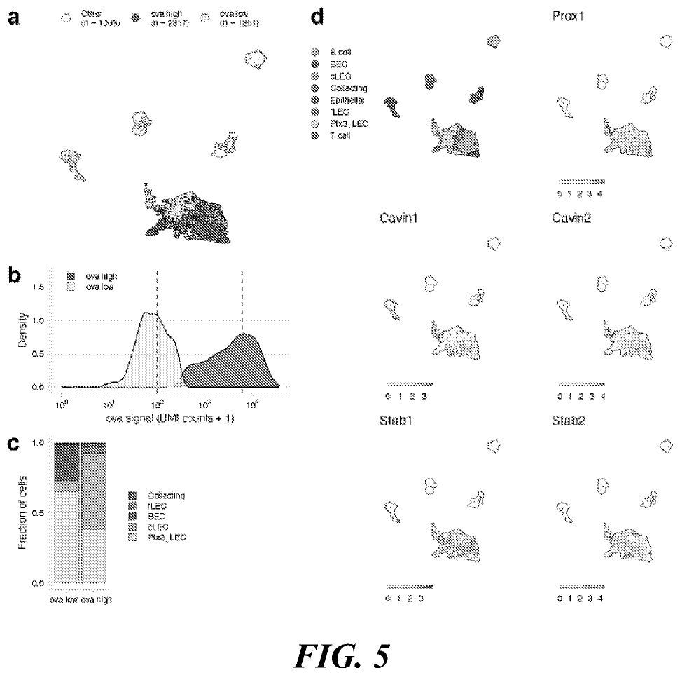

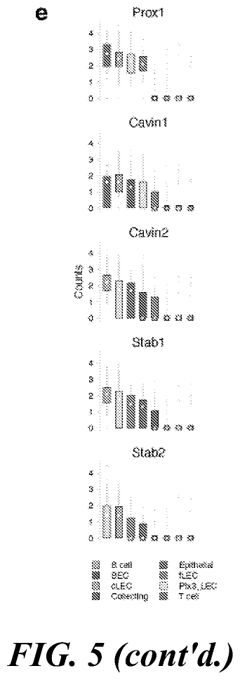

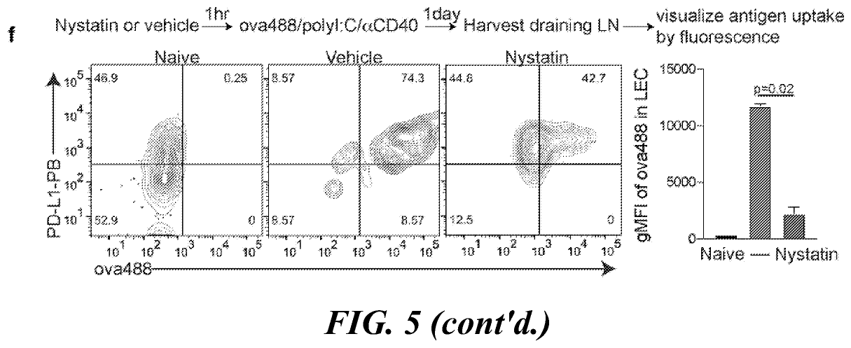

Identification of ova-low and -high populations was accomplished using a two-component Gaussian mixture model implemented with the R package mixtools (cran.r-project.org/web/packages/mixtools/index.html). All LECs were used when identifying ova-low and ova-high cells (FIG. 5). For DCs (FIG. 4), ova-low and -high populations were identified independently for each DC cell type. For ova-low and ova-high populations, differentially expressed genes were identified using the R package presto (wilcoxauc, github.com/immunogenomics/presto). Differentially expressed genes were filtered to include those with an adjusted p-value<0.05, log fold change>0.25, AUC>0.5, and with at least 50% of ova-high cells expressing the gene.

Raw data and analysis software. Raw and processed data for this study have been deposited at NCBI GEO under accession GSE150719. A reproducible analysis pipeline is available at github.com/rnabioco/antigen-tracking.

Statistical analysis. Statistical analysis was done using either a non-parametric 2 tailed Mann-Whitney t-test or multiple t-tests with a two stage step up method of Benjamin, Krieger and Yekutieli without assuming consistent standard deviations. A biologic replicate was considered a measurement of a biologically distinct sample (such as a separate mouse), a technical replicate was considered a repeated measurement of the same sample. Each in vivo analysis was performed with 3-6 mice per group as determined by a power calculation using the assumption (based on prior data) that there will be at least a two-fold change with a standard deviation of less than 0.5. To calculate numbers, a power calculation was performed with an alpha of 0.5 and a 1-beta of 0.80 to determine at least 3 mice per group should be evaluated. Error bars indicate the standard error of the mean (SEM) and all analysis was blinded.

### Example 2: Results

### Generation, Validation, and Immunogenecity of Antigen-DNA Conjugates.

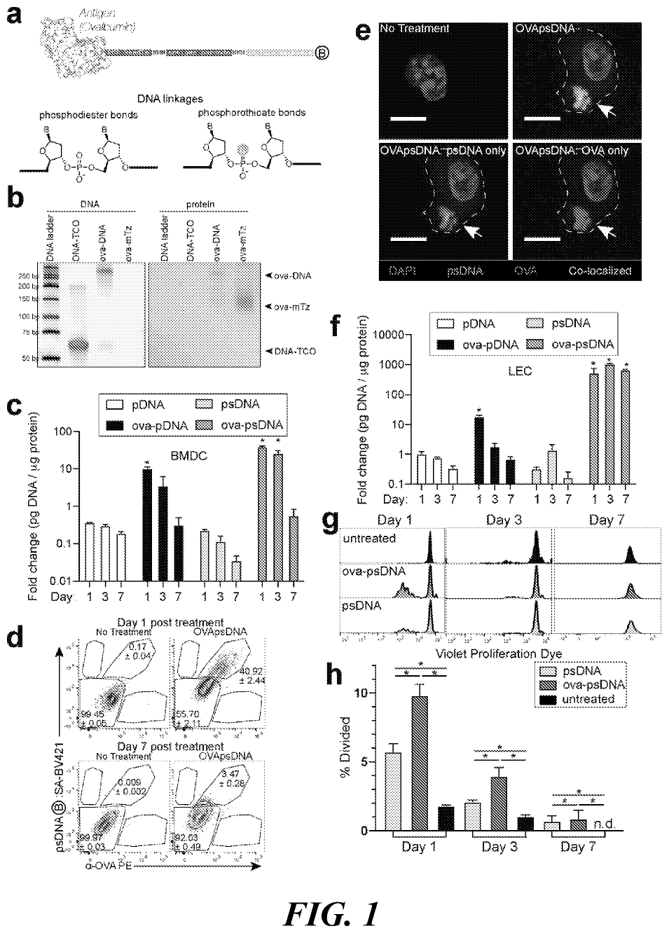

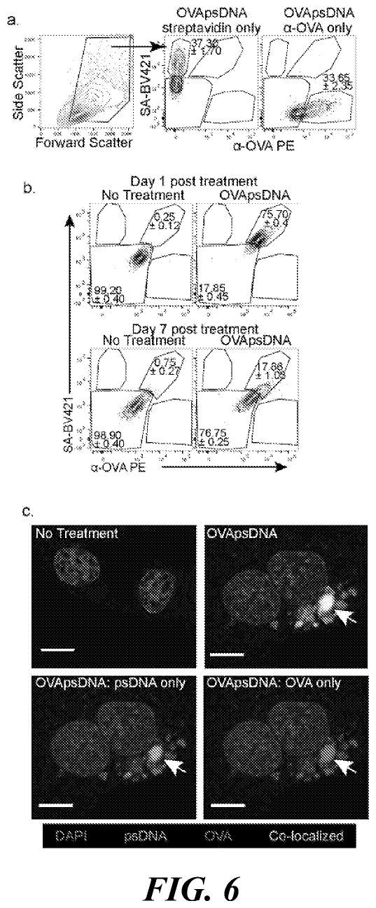

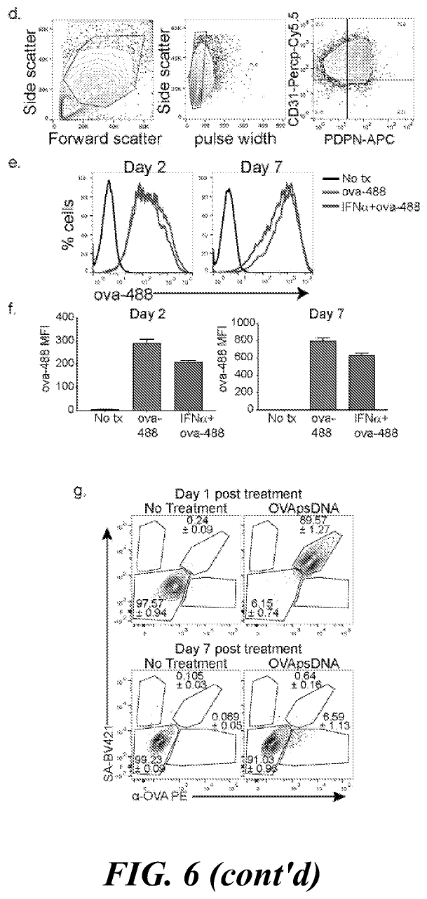

To quantify the dissemination and uptake of antigen in the draining lymph node (LN) after vaccination, a strategy was developed to measure antigen levels using single-cell mRNA sequencing. Many prior studies have used the model antigen, ovalbumin (ova), conjugated to a fluorophore to track antigen in vivo. Here, ova to DNA oligonucleotides with barcodes were conjugated that were suitable for analysis by single-cell mRNA sequencing (FIG. 1, panel a). The ˜60 nt DNA tag contains a unique sequence barcode and PCR primer binding sites, similar to CITE-seq tags⁴⁰ (Supplementary Table 1 from Walsh et al.). The stability of unconjugated DNA and ova-DNA conjugates was measured in which the conjugated DNA either had normal phosphodiester linkages (pDNA) or was protected throughout by phosphorothioate linkages (psDNA). Quality control of these conjugates indicated a 1:1 stoichiometry of protein to DNA (FIG. 1, panel b). To measure the stability of the antigen-DNA conjugate, antigen-DNA conjugates were added to cultures of bone marrow derived dendritic cells (BMDCs) and the amount of DNA in cell lysates and media was quantified over time. It was found that significantly higher levels of ova-psDNA in cells relative to ova-pDNA (˜4-fold at day 1; p=0.002 and ˜7 fold at day 3; p=0.004), indicating that psDNA is more stable than pDNA, but is processed by BMDCs at the same rate as ova protein (FIG. 1, panel c). In addition, ova conjugation was required for phagocytosis by BMDCs, as limited amounts of unconjugated pDNA or psDNA were detected (values<1 at days 1-7) (FIG. 1, panel c). To determine if the BMDCs had both the ova and DNA within the cell, flow cytometry and immunofluorescence was performed using an antibody to detect ova and streptavidin to detect the biotinylated DNA tag. Both ova and DNA was detected within the same cells by flow cytometry (FIG. 1, panel d, FIG. 6, panel a) and co-localization by immunofluorescence (FIG. 1, panel e). Conjugate stability in mouse LECs was also measured, a cell type that retains foreign proteins for long periods¹⁴, and it was found that ova-psDNA conjugates were stable over 7 days of culture, whereas ova-pDNA was rapidly degraded (FIG. 1, panel f). In the endothelial cells, both the ova protein and the barcode were detected within the same cell and co-localized to the same location (FIG. 6, panels b, c). Furthermore, the ova-psDNA retention within the LECs was similar to a vaccine strategy using an ova protein-fluorophore conjugate with polyl:C and anti-CD-40, which was previously demonstrated to induce antigen archiving⁴¹ (FIG. 6, panels d-f). Using a more phagocytic cell, bone marrow-derived macrophages, nearly all macrophages were observed to phagocytose the ova-psDNA at day 1 and found the ova and psDNA within the same cell (FIG. 6, panel g). In macrophages given ova-psDNA 7 days prior, only ova protein was detected (FIG. 6, panel g), potentially resulting from high levels of endonucleases found within the lysosome of macrophages.

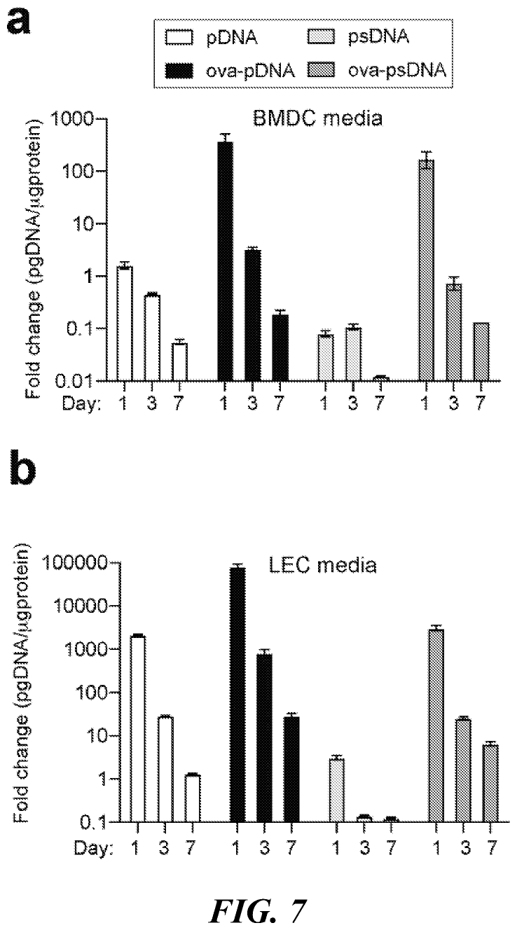

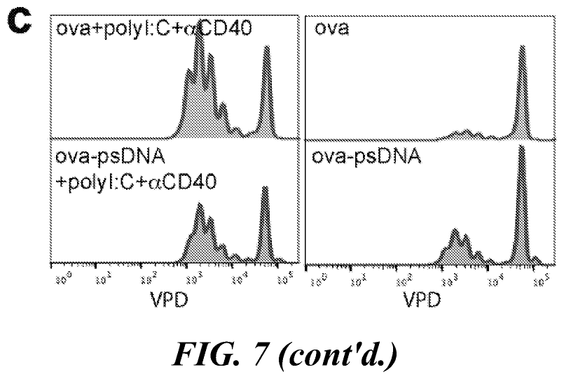

To determine whether conjugation of psDNA to ovalbumin affected ovalbumin processing and presentation, BMDC presentation of ova-derived SIINFEKL (SEQ ID NO:1, OVA257-264) peptide was measured by co-culture with OVA257-264-specific OT1 T cells. BMDCs given ova-psDNA induced significantly more proliferation of OT1 T cells than unconjugated ovalbumin (FIG. 1, panels e, f) suggesting enhanced activation of BMDCs upon encounter with ova-psDNA conjugates. Furthermore, pDNA and psDNA was detected in BMDC culture media at one day after addition but not at later time points, confirming that ova-psDNA conjugates are processed and not released by BMDCs after phagocytosis (FIG. 7, panels a, b). Finally, ova-psDNA conjugates led to increased OT1 proliferation at day 3 relative to ova plus psDNA (unconjugated), showing that ova-psDNA conjugates are immunostimulatory (FIG. 1, panels g, h) and consistent with studies showing conjugation of antigens to RNA or DNA induce TLR7 (RNA) or TLR9 (DNA) signals that lead to prolonged antigen presentation⁴¹. Addition of polyI:C and anti-CD40 to BMDCs with ova also elicited robust OT1 proliferation, demonstrating that TLR activation on the BMDCs is required for efficient cross-presentation to T cells (FIG. 7, panel c).

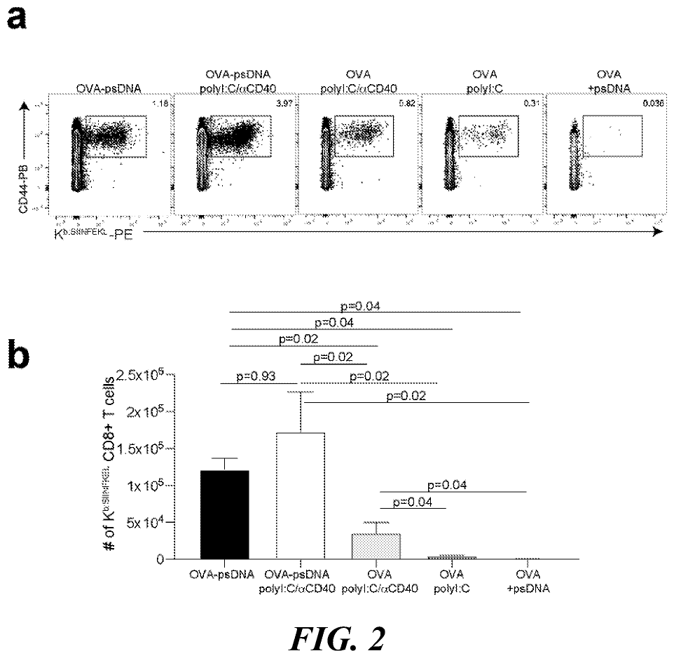

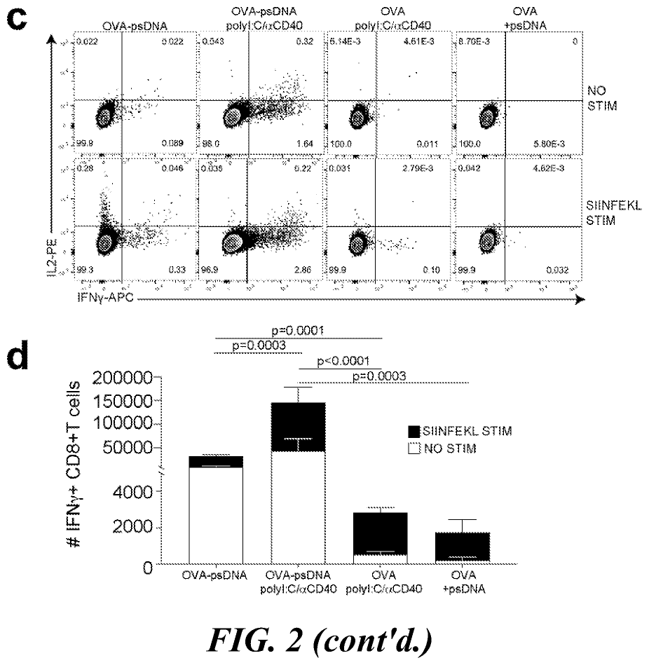

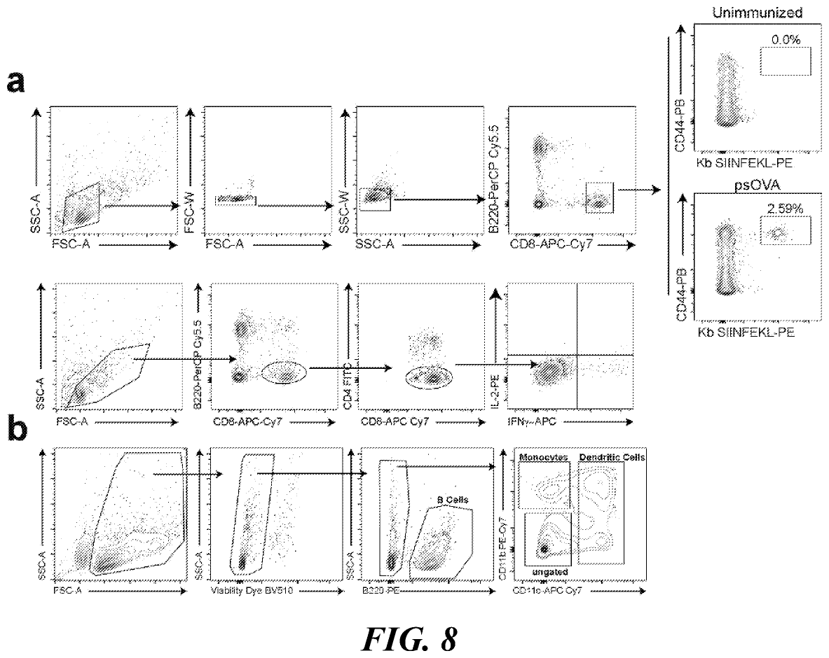

It was next asked whether vaccination with ova-psDNA conjugates elicits a T cell response in vivo. Antigen-specific T cell responses were compared in mice vaccinated with a mixture of ova-psDNA and polyI:C/αCD40 to its individual components (ova, psDNA, polyI:C and polyI:C/αCD40; FIG. 2, panel a and FIG. 8, panel a) and—consistent with the differences seen in OT1 proliferation in vitro—found that T cell responses to ova-psDNA were greater than either ova with polyI:C, ova with polyI:C/αCD40, or a mixture of unconjugated ova and psDNA (FIG. 2, panel b). Interestingly, ova-psDNA conjugate combined with polyI:C/αCD40 did not significantly enhance the T cell response beyond ova-psDNA alone (FIG. 2, panel b). T cells stimulated by ova-psDNA produced significantly more IFNγ than any other vaccination strategy even in the absence of ex vivo OVA257-264 peptide stimulation, indicating prolonged and active presentation of ova-psDNA (FIG. 2, panels c, d). Together these data show that ova-psDNA conjugates elicit antigen-specific T cell responses independent of polyI:C/αCD40. These findings are consistent with TLR9-dependent immune responses elicited by psDNA^{42,43,44}, similar to DC presentation of conjugates of ova demonstrated with other TLR agonists⁴⁵ and other subcutaneously administered ova-TLR conjugate vaccine platforms⁴¹.

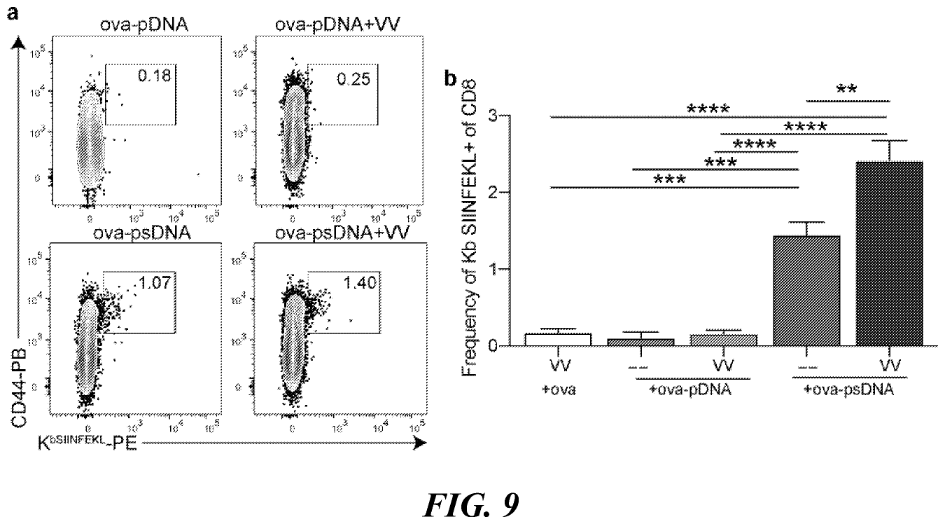

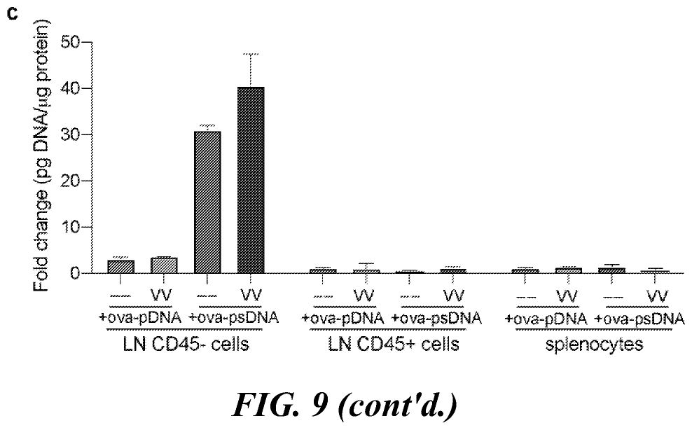

It has been previously shown that a vaccination strategy comprised of soluble antigen and vaccinia virus (Western Reserve; VV) induced robust antigen archiving that lasts longer than those using polyI:C/αCD40 adjuvant¹². To evaluate antigen-psDNA performance during an active infection, T cell responses after vaccination were determined by comparing individual components with mixtures of ova, VV, ova-pDNA, or ova-psDNA. Subcutaneously administered ova-psDNA alone again elicited a T cell response (FIG. 2 and FIG. 9, panel a) and addition of VV to ova-psDNA conjugate moderately increased T cell responses compared to ova-psDNA alone, similar to what was observed with ova-psDNA/polyI:C/αCD40 (FIG. 2 and FIG. 9, panel b). Finally, the cell type-specificity of ova-psDNA dissemination in vivo was examined. Mice were vaccinated with mixtures of (i) ova-psDNA and VV or (ii) ova-psDNA and polyI:C/αCD40, and levels of ova-psDNA were quantified by PCR in both leukocytes and stromal cells (fractionated by CD45 expression) in the draining lymph nodes. It was found that CD45− stromal cells had high amounts of ova-psDNA, corresponding to increased inflammation¹⁴, whereas CD45+ leukocytes had very low levels of ova-psDNA 20 days after vaccination/infection (FIG. 9, panel c). These data recapitulate a previous demonstration of durable antigen retention by CD45− stromal cells^{12,14}, confirming that ova-psDNA is a faithful tracking device for antigen archiving in vivo.

### Molecular Tracking of Antigen During the Immune Response to Vaccination

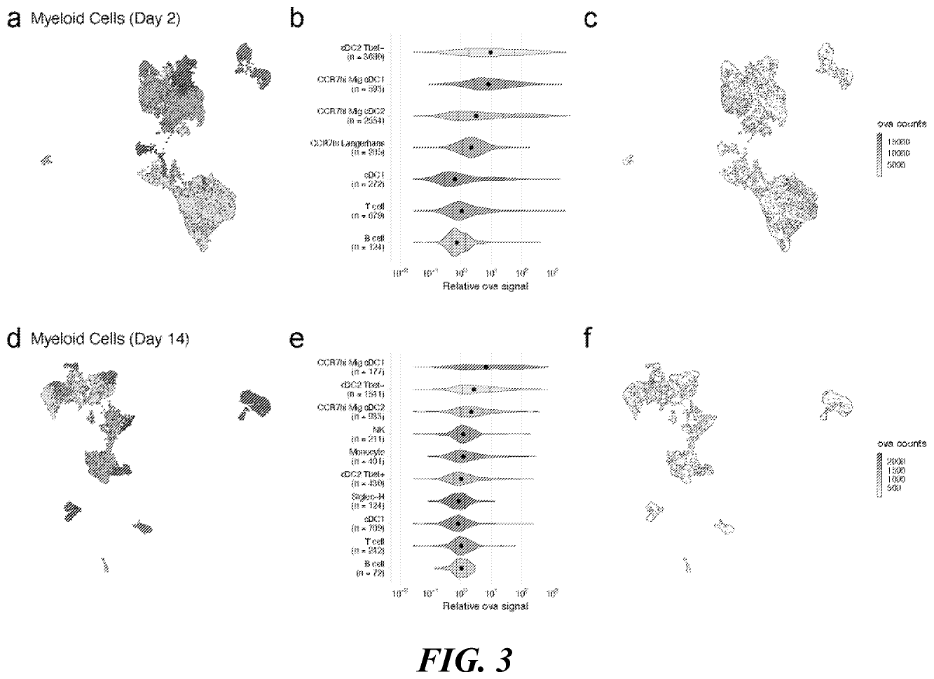

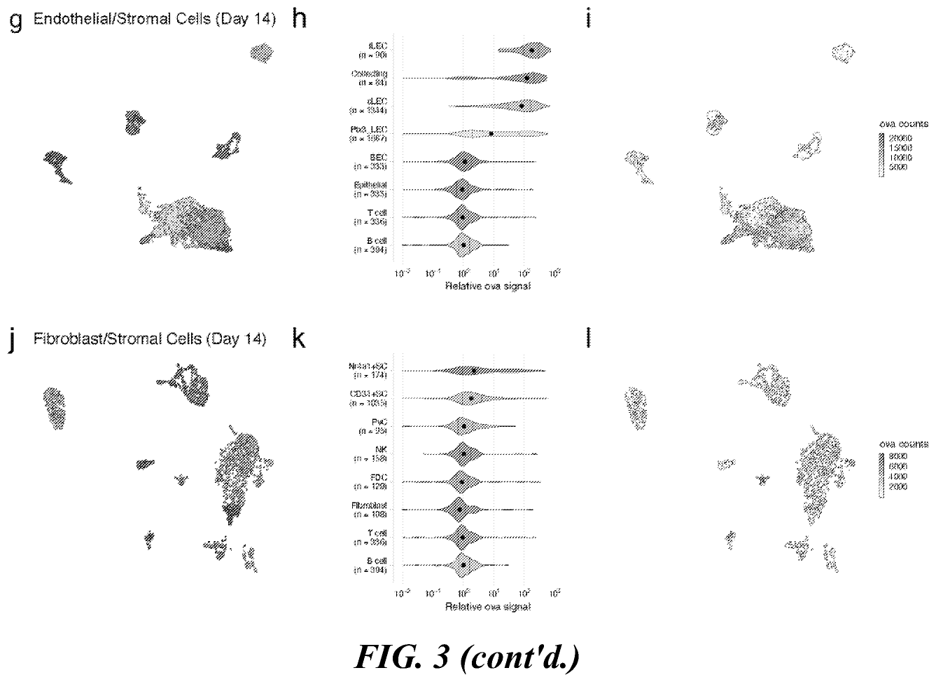

Given the ability of the antigen-psDNA conjugates to induce a robust immune response in vivo (FIG. 2) and the ability to use the psDNA as a measure of protein antigen levels (FIG. 1), the antigen-psDNA conjugates were used as a “molecular tracking device” to understand the distribution of the protein antigen in the lymph node following vaccination. To determine whether cells could be identified that acquire and archive antigens¹⁴, mice were vaccinated subcutaneously with an equimolar mixture of uniquely barcoded ova-psDNA conjugate, unconjugated psDNA, and unconjugated pDNA (unprotected phosphodiester backbone) with VV (as in FIG. 9, panel c), and evaluated antigen distribution (via psDNA abundance) in the lymph node at early (2 days) and late (14 days) time points. At each time point, single-cell suspensions were prepared from draining popliteal lymph nodes and divided into stromal cell (by depleting CD45+ cells) or lymphocyte populations (by flow sorting for CD11c, CD11b, and B220 markers; FIG. 8, panel b). To enrich for myeloid cell populations but maintain representation of other cell types; CD11c+, CD11b+, B220+, and ungated live cells were mixed at a 4:4:1:1 ratio, respectively. These cell populations were analyzed by single-cell mRNA sequencing, measuring both mRNA expression and the quantity of psDNA in each cell using unique molecular identifiers⁴⁶ (FIG. 3).

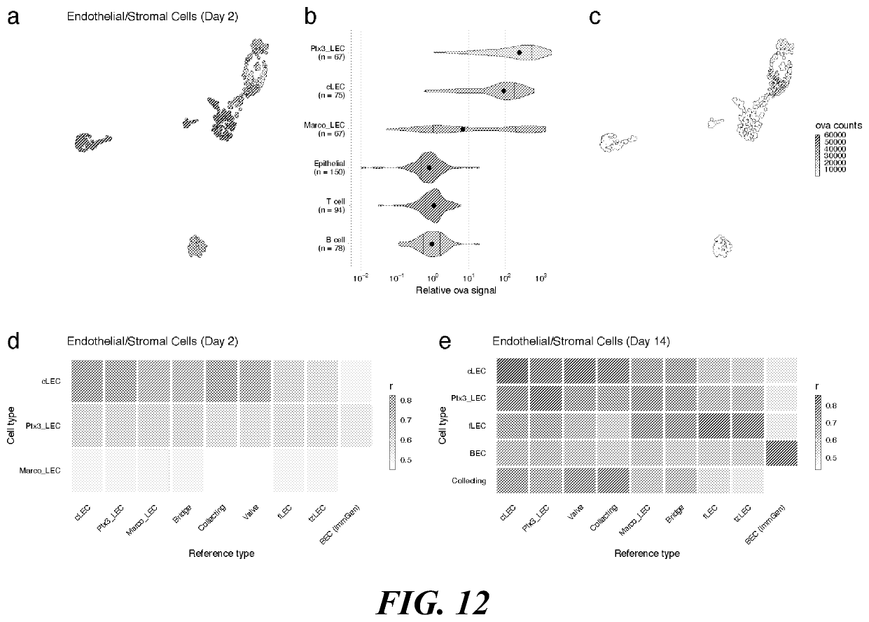

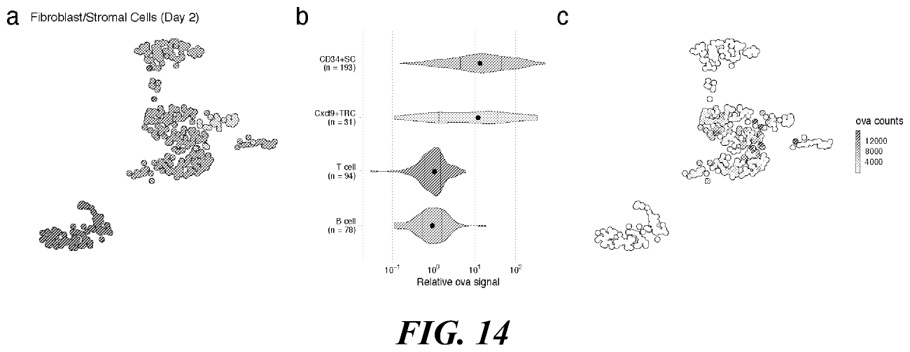

A total of 800 cells were recovered in the CD45− fraction and 8,187 cells in the CD45+ fraction at the 2 day time point. More CD45− cells (6,372 CD45−; 4,840 CD45+) were recovered at the 14 day time point likely due to expansion and proliferation of the lymph node stoma^{14,22,47}. Cell types were classified using an automated approach⁴⁸, comparing measured mRNA expression patterns to reference data sets for DCs^{49,50}, FRCs⁵¹, and LECs^{52,53,54} (Supplementary Table 2 from Walsh et al. doi.org/10.1101/2020.08.19.219527v1 (2020), the disclosure of which is incorporated herein by reference). Supplementary Table 2 from Walsh et al. lists the results from comparing relative ova signal for cell types shown in FIG. 3, FIG. 12, panel b, and FIG. 14, panel b. A two-sided Wilcoxon rank sum test was used to compare relative ova signal for each cell type. Relative ova signal was calculated by dividing antigen counts for each cell by the median antigen counts for T and B cells. The Bonferroni method was used to correct for multiple comparisons. The number of cells in each group (n cells), fraction of the total cells for the sample (frac cells), median relative ova signal, test statistic (statistic), estimation of the median difference (estimate), and confidence interval (conf low, conf high) are included. As expected, the CD45+ fraction contained DCs, monocytes, T cells, and B cells (FIG. 3, panels a, b, d, e), while the CD45− fraction contained stromal cells (SC), including LECs, blood endothelial cells (BEC), epithelial cells, and fibroblasts (FIG. 3, panels g, h, j, k). Vaccinia viral mRNAs were not recovered in cells at either time point, possibly due to viral clearance or a failure to recover infected, apoptotic cells in the live/dead selection (FIG. 8, panel b).

The dynamic changes of myeloid populations were firs examined in the lymph node. Conventional DCs were detected, including cDC1 and cDC2 (FIG. 3, panels a-c), which develop from a common DC precursor upon expression of FMS-like tyrosine kinase 3 ligand (Flt3L)⁵⁵. Lymph node (LN) resident and migratory cDCs can be distinguished by expression of cell-type-specific transcription factors including basic leucine zipper transcription factor (Batf3) and interferon regulatory factor (IRF8) (cDC1)^{56,57,58}or IRF4 and Notch (cDC2)^{59,60}. These cDC types are also typically classified based on expression of CD11c, Zbtb46, and chemokine XC receptor 1 (cDC1 are XCR1+, cDC2 are XCR1−)^{55,61}. cDC2s are further categorized as either Tbet-dependent and anti-inflammatory (cDC2A) or RORγt-dependent and pro-inflammatory (cDC2B)⁴⁹.

As expected, at day 2 a large population was identified of LN resident cDC2B (cDC2 Tbet−) cells harboring ova-psDNA⁴⁹. However, cDC2A (cDC2 Tbet+) cells were not found, consistent with their role in anti-inflammatory processes⁴⁹. The myeloid populations contained CCR7^{hi} cDCs (n=3,432; 42% of total), which were classified as migratory DCs. This migratory DC population included Langerhans cells (n=285; 3.5% of total), migratory cDC1s (n=593; 7.2% of total), and migratory cDC2s (n=2,554; 31% of total) 50 , migrating from the dermis (FIG. 3, panel b). At day 14, a population of LN-resident cDC2 Tbet+ cells was identified (FIG. 3, panel e), consistent with resolution of the immune response⁴⁹. As cDC2 Tbet+ cells are thought to be anti-inflammatory, these data indicate that the immune response is being quelled (FIG. 3, panel e). A group of Siglec-H+ DCs, a cDC progenitor population⁴⁹ (FIG. 3, panels d, e) was also found.

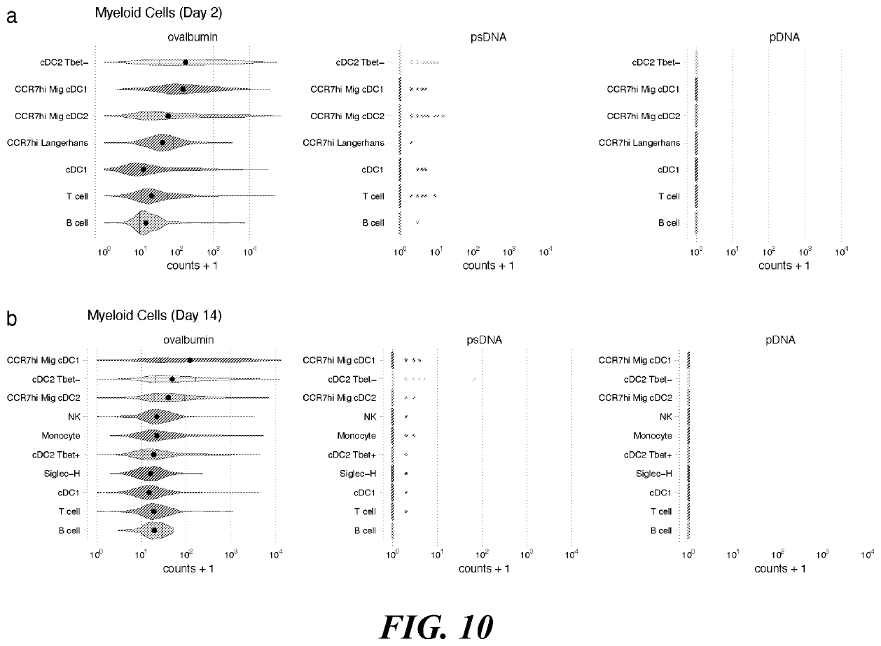

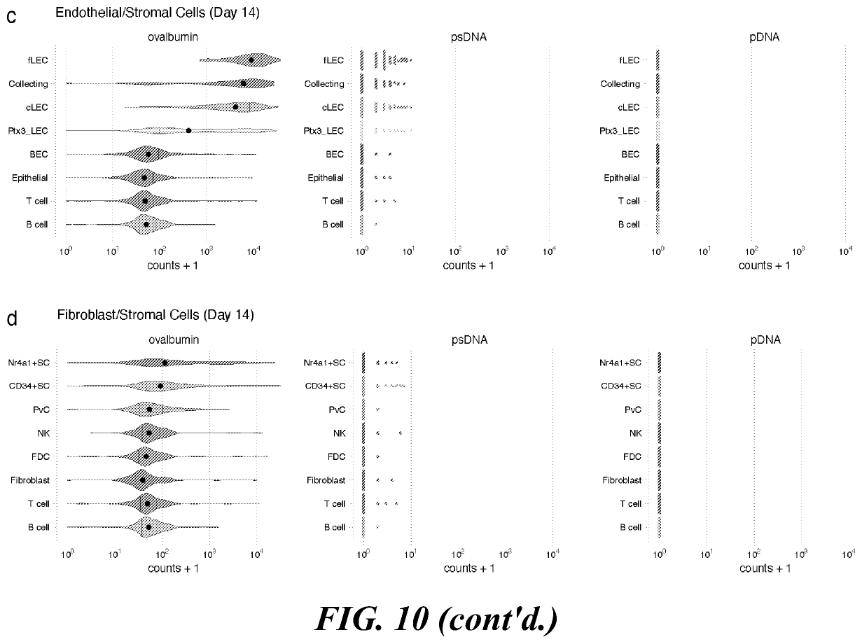

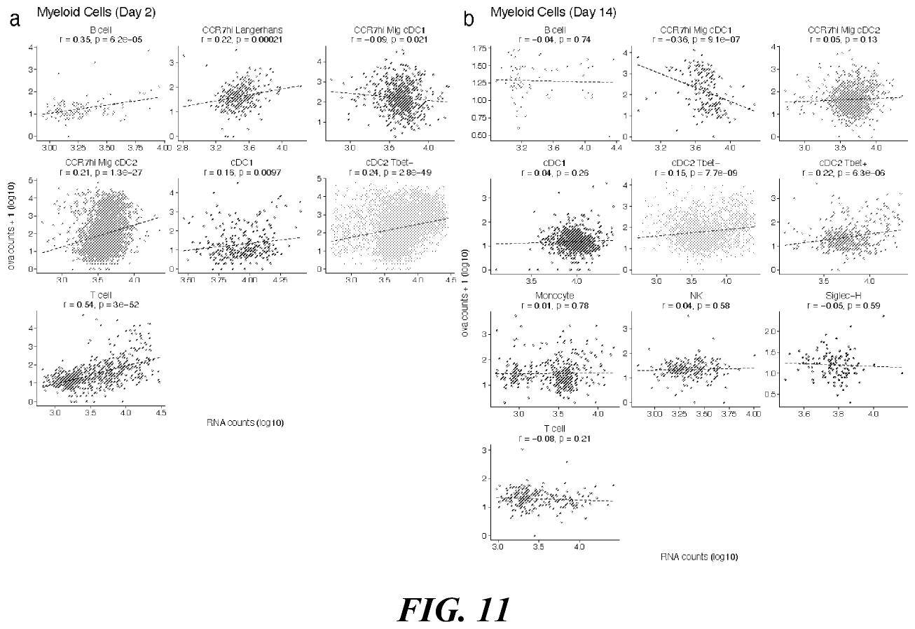

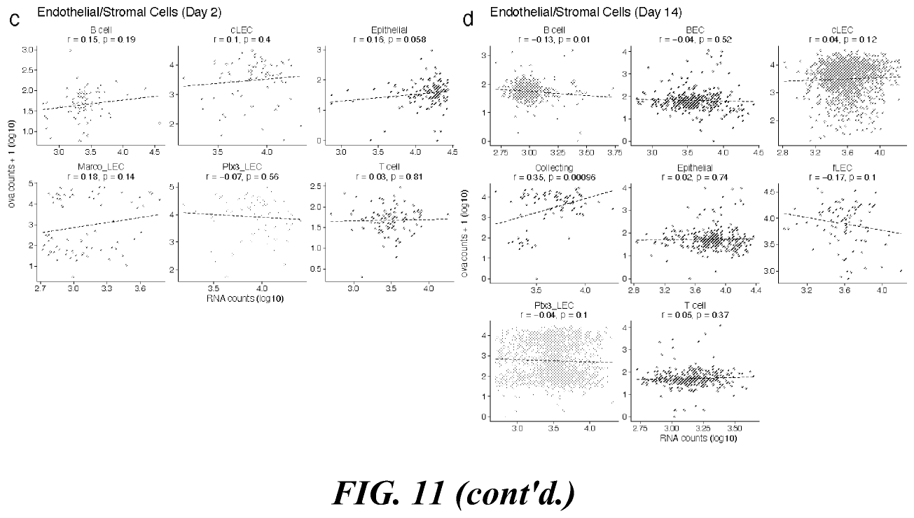

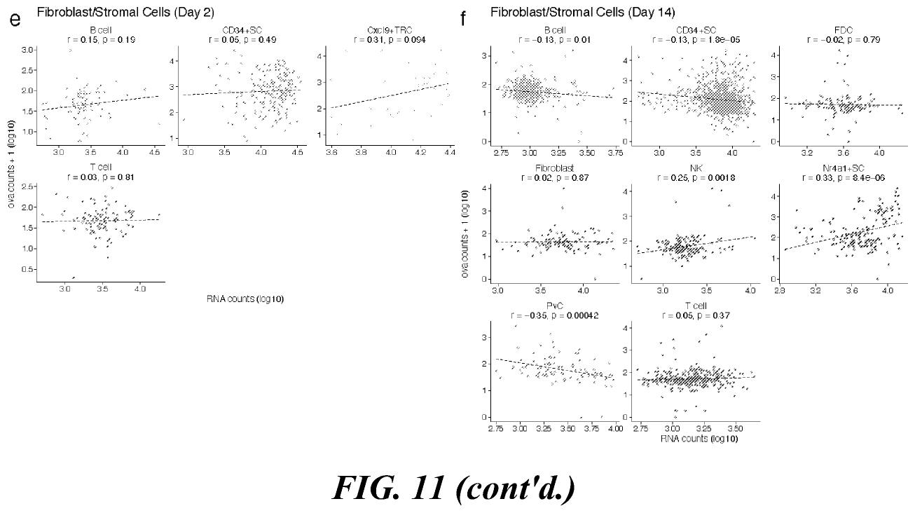

Using unique barcodes, the amount of ova-psDNA, psDNA, and pDNA across cell types was quantified. Levels of ova-psDNA molecules spanned four orders of magnitude, ranging up to 10⁴ unique molecules and depending on the cell types and time point (FIG. 3, panels c, f, i, l). In contrast to the large range of ova-psDNA across cell types, unconjugated psDNA and pDNA were largely undetectable, indicating that antigen conjugation is required for cell acquisition (FIG. 10). Consistent with previous studies¹², antigen-psDNA was not detected at appreciable levels in T cells or B cells (FIG. 3) and because these cell types were captured in both CD45− and CD45+ samples, their median antigen levels were used to normalize antigen counts in other cell types across captures. The trivial case was considered wherein variation in antigen levels is explained by total mRNA abundance; these variables are uncorrelated in stromal cell types and weakly correlated in cDC subtypes, possibly reflecting activation status (FIG. 11).

At the early day 2 time point, LN resident cDC2s contained high levels of antigen-psDNA, consistent with studies of soluble antigens² (FIG. 3, panels b, c). In addition, significantly higher levels of antigen in cDC2 Tbet−, migratory CCR7^{hi} cDC2s, and migratory CCR7^{hi} cDC1s were found (FIG. 3, panels b, c; Supplementary Table 2 from Walsh et al.), with an average of ˜7-fold more antigen than T/B cells. At the later time point, migratory cDC1 cells contained the most antigen, consistent with previous studies¹² (FIG. 3, panel e). In addition, Tbet− and CCR7^{hi} migratory cDC2s contained moderate levels of antigen, up to 3-fold more than T/B cells, but had lower amounts of antigen relative to day 2 (FIG. 3, panels b, c, e, f; Supplementary Table 2 from Walsh et al.). At the late time point, significant amounts of antigen in LN resident cDC1s, Tbet+ cDC2s, Siglec-H+ cells, or monocytes were not detected (FIG. 3, panel e).

Next, antigen levels in the LN stromal cell populations were examined (FIG. 3, panels g-l). Endothelial cells in the lymph node are classified by their association with blood or lymphatic vasculature; both are required for circulation and trafficking of immune cells to the lymph node. The blood vasculature circulates naïve lymphocytes to the LN and the lymphatic vasculature transports immune cells from the peripheral tissue including dermal DCs and memory T cells. An automated approach⁴⁸ was used, that uses correlation between reference and measured gene expression profiles to assign unknown cell types to subtypes defined by previous studies. While strong correlation reflects a good match between reference and query profiles, high correlation between multiple reference LEC subtypes^{52,53,54}, and changes in expression induced by antigen acquisition made definitive cell type assignments challenging (FIG. 12, panels b-d). Notwithstanding these issues, LEC subsets were classified based on the highest correlation values to reference cell types (FIG. 12, panels d, e)⁵⁴ and identified three LEC subtypes^{52,53,54} including Ptx3 LECs, ceiling LECs, and Marco LECs with high levels of antigen at the early time point (FIG. 12, panel a). At the late time point, expansion and proliferation of lymph node stromal cells contributed to larger populations of cells including floor LECs, collecting LECs, ceiling LECs, Ptx3 LECs⁵³, and blood endothelial cells (BECs) (FIG. 3, panel h)⁶².

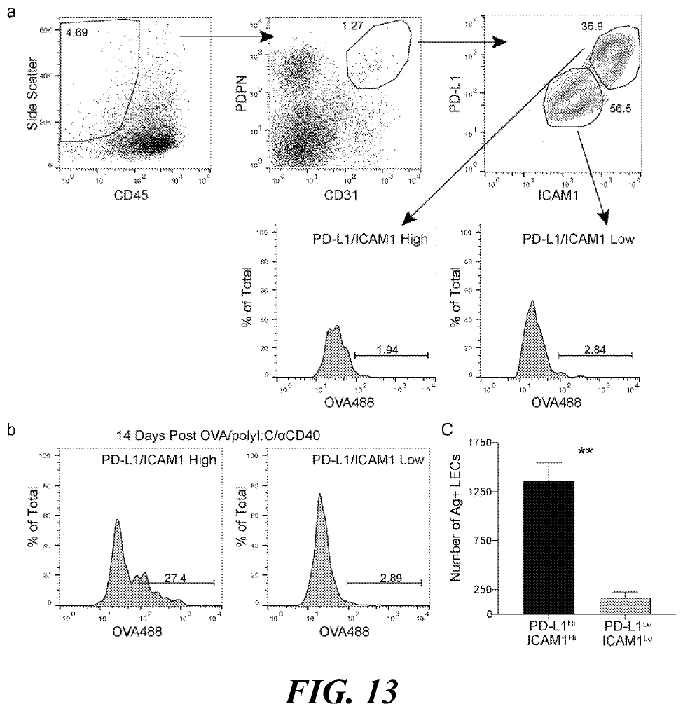

At the day 14 time point, several LEC subtypes maintained high antigen levels (FIG. 3, panel h, Supplementary Table 2 from Walsh et al.). Floor LECs had uniformly high amounts of antigen, confirming previous studies using flow cytometric analysis of fluorescent antigen¹². Median levels of antigen in collecting, Ptx3, and ceiling LEC populations were significantly higher than B/T cells, but cells in these groups contained a range of antigen with both high and low populations. It was hypothesized this variability stems from the physical location of the LECs within the LN and their access to trafficking antigen, and confirmed that fluorescent antigen amounts are highest on subcapsular LECs as identified by surface expression of PD-L1 and ICAM1 two weeks after immunization^{22,32} (FIG. 13). Together these findings suggest that antigen first passes through the sinus followed by the cortex and medulla. These data also suggest that populations of LECs with less antigen could be a result of how the antigen travels through the lymph node or mechanisms of antigen release over time.

Similar to the endothelial cell population, the number and types of non-endothelial stromal cells increased at the later time point after immunization. Non-endothelial stromal cells in the lymph node are classified by their location in the lymph node into T-zone reticular cells (TRC), marginal reticular cells (MRC), follicular dendritic cells (FDC), and perivascular cells (PvC)⁵¹. Recently, additional subsets were identified including: Ccll9^{lo} TRCs located at the T-zone perimeter, Cxcl9⁺ TRCs found in both the T-zone and interfollicular region, CD34⁺ stromal cells found in the capsule and medullary vessel adventitia, indolethylamine N-methyltransferase⁺ stromal cells found in the medullary chords, and Nr4a1⁺ stromal cells⁵¹.

At the early time point, the Cxcl9+ TRCs and CD34+ SCs⁵¹, had high amounts of antigen (˜10-fold relative to TB cells) (FIG. 14 and Supplementary Table 2 from Walsh et al. doi.org/10.1101/2020.08.19.219527v1 (2020)). At the late time point, CD34+ SCs, Nr4a1+ SCs, follicular dendritic cells (FDC), and PvCs were detected (FIG. 3, panel k). Only the CD34+ and Nr4a1+ SCs contained significant amounts of antigen (FIG. 3, panel k, Supplementary Table 2 from Walsh et al.). Interestingly, the CD34+ stromal cells are adjacent to ceiling LECs and the Nr4a1+ SCs are found in the medullary chord and medullary sinus, which are lined by medullary LECs. These findings may suggest potential antigen exchange mechanisms between LECs and SCs that have yet to be defined. Little antigen in PvCs or FDCs was found (FIG. 3, panel k).

Finally, these data provided insight into antigen transfer between stromal and dendritic cells, a process important for enhanced protective immunity^{12,14}. It was previously shown that archived antigen is transferred from LECs to migratory Batf3-dependent cDC1s two weeks after infection¹². Here, it was confirmed that CCR7^{hi} migratory cDC1s had the highest amount of antigen two weeks after vaccinia infection (FIG. 3, panel e)¹². Together, these data validate the use of molecular tracking devices by corroborating previous studies of antigen trafficking and identify new cell types that dynamically acquire antigen during infection.

Gene Expression Signatures Associated with Antigen Acquisition by Dendritic Cells.

Next, the variation in antigen levels across cell types was leveraged (FIG. 3, panels b, e, h, k) to identify gene expression signatures associated with high levels of antigen that would validate this approach. Cells were classified as “antigen-high” and “antigen-low” using a two-component mixture model, and identified marker genes associated with each class (FIG. 4, panels a, b). To validate this approach, the dendritic cell populations were evaluated, as genes associated with phagocytosis and activation have been established^{50,63,64,65,66,67,68,69,70,71,72}. Dendritic cell populations generally contained lower antigen levels that were variable across subtype (FIG. 3). Antigen-low and antigen-high cells were classified for each subtype. Among the subtypes with significant amounts of antigen, Tbet− cDC2 cells had the highest antigen levels and largest differences in gene expression (277 genes in antigen-high cells, FIG. 4, Supplementary Table 4 from Walsh et al. doi.org/10.1101/2020.08.19.219527v1 (2020), the disclosure of which is incorporated herein by reference), consistent with cDC2s acting as the primary cell type of antigen uptake following protein immunization². Supplementary Table 4 from Walsh et al. lists genes associated with ova-low and ova-high cells forDC, FRC, and LEC cell types. Ova-low and ova-high cells were identified independently for each cell type shown in FIG. 3 using a gaussian mixture model implemented with the R package mixtools. Differentially expressed genes were identified as described for Supplementary Table 3 from Walsh et al. doi.org/10.1101/2020.08.19.219527v1 (2020), the disclosure of which is incorporated herein by reference.

At the early time point, genes upregulated in antigen-high DCs confirmed DC activation (Supplementary Table 4 from Walsh et al.). Antigen-high cDC2 Tbet− cells upregulated genes Ccl2 and Cxcl2 (consistent with active recruitment of inflammatory cells^{66,69}), Msr1 (consistent with antigen scavenging⁷¹), as well as Pkm, Lgals3, and Mif (consistent with DC-T cell responses and DC differentiation during inflammation^{36,65,68}) (FIG. 4, panels c, d).

At the late day 14 time point, the highest antigen counts were found in the migratory cDC1 population, consistent with a role for migratory cDC1s in archived antigen acquisition from LECs¹² (FIG. 3, panels e, f). Among the genes highly expressed by the antigen-high CCR7^{hi} migratory cDC1 population were Ccl5 and Fscn1 (Supplementary Table 4 from Walsh et al.). Consistent with these DCs being involved in archived antigen presentation, Ccl5/RANTES regulates CD8 T cell responses during chronic viral infection⁷³ and Fscn1, an actin binding protein, regulates cell migration of mature DCs via podosome formation⁷⁴. Similar to the day 2 timepoint, the differences in gene expression for antigen high and low cells were greatest within for Tbet− cDC2 populations (230 genes in antigen-high cells; FIG. 4, panels e, f, and Supplementary Table 4 from Walsh et al.). Genes upregulated in antigen-high tbet− cDC2s included Fcgr4, which is involved in phagocytosis, antigen presentation, and proinflammatory cytokine production^{67,70}, and CD72 and Acp5, which are important for the inflammatory response and pathogen clearancez^{64,72} (FIG. 4, panels g, h). Collectively, these genes evoke specific processes in DC subsets required for the immune response; it remains to be determined whether they are specifically associated with LEC-DC antigen exchange or storage of antigens within DCs.

Gene Expression Signatures Associated with Antigen Archival by LECs.

Next, the LEC population was evaluated to determine whether the classification approach could identify genes involved in antigen archiving. The classifier was applied to LECs as a population and found large numbers of antigen-high-floor, collecting, and ceiling LECs (FIG. 5, panel c). Ptx3 LECs were comprised of a mixture of antigen-low and antigen-high cells, but there was a larger fraction of Ptx3 LECs with low antigen (FIG. 5, panel c). There were less antigen-low LECs compared to antigen-high LECs overall (34% of total), suggesting that antigen archiving may be specific to LECs in general rather than attributable to a specific LEC subset (FIG. 5).

Using this classification approach, 142 mRNAs were identified that were significantly changed in antigen-high or antigen-low LECs (Supplementary Table 3 from Walsh et al.). Supplementary Table 3 from Walsh et al. lists genes associated with ova-high cells for DCs, FRCs, and LECs. Ova-low and ova-high cells were independently identified for DCs, FRCs, and LECs using a gaussian mixture model implemented with the R package mixtools. Differentially expressed genes were identified using a Wilcoxon rank sum test performed using the R package presto (wilcoxauc). The Benjamini-Hochberg method was used to correct for multiple comparisons. Genes were filtered to only include those with an adjusted p-value<0.05, log fold change>0.25, auc>0.5, and with at least 50% of ova-high cells expressing the gene. The average expression, log fold change, test statistic (statistic), area under the receiver operator curve (auc), percentage of ova-high cells that express the gene (pct_in), and percentage of ova-low cells that express the gene (pct out) are included. Prox1, while expressed by all LECs identified, was highly expressed in antigen-high LECs, independent of the LEC type (FIG. 5, panels d, e). Prox1 is a transcription factor required for LEC differentiation from blood endothelial cells and defines LEC identity via regulation of Vegfr3, Pdpn, and Lyve-1^{75,76,77}. Prox1 upregulation in antigen-high LECs indicates it may also transcriptionally regulate processes involved in antigen archiving.

Upregulation of Cavin1 and Cavin2 by antigen-high LECs suggested that caveolar endocytosis may contribute to antigen acquisition by LECs, consistent with LEC dynamin-mediated transcytosis in vitro⁷⁸ (FIG. 5, panels d, e). Cavin2 appears more specific to LECs than Cavin1, which is also upregulated by BECs, suggesting that Cavin2 mediates endocytosis specifically in endothelial cells of the lymphatic lineage. Based on Cavin2 gene expression it appears this process may be most active in ceiling LECs (FIG. 5, panel e). To confirm this finding, it was asked whether inhibition of the caveolin pathway with nystatin impaired endocytosis of fluorescent antigen in mice vaccinated with ovalbumin/polyI:C/αCD40. It was found that a significant decrease in antigen acquired by LECs in the nystatin treatment group 24 hours after administration of fluorescent antigen with an innate immune stimulus (FIG. 5, panel f), affirming the utility of molecular tracking devices for identifying genes involved in the process of antigen acquisition or archival that are not necessarily specific to antigen-psDNA conjugates.

Finally, expression of Stabilin-1 (Stab1) and Stabilin-2 (Stab2) is increased in antigen-high LN endothelial cells, suggesting that scavenging pathways are required for the acquisition of antigen-psDNA conjugates after vaccination. Stab2 is uniquely expressed by LECs in the lymph node and not by BECs⁷⁹, and Stabilin-1 and Stabilin-2 act as receptors for internalization of antisense oligonucleotides with phosphorothioate linkages in liver endothelial cells and Kupffer cells⁸⁰. However, significant amounts of unconjugated psDNA were not found in LECs (FIG. 10), indicating that Stab 1/Stab2 are upregulated as part of an antigen scavenging or trafficking program initiated in LECs upon antigen acquisition during infection.

### Example 3: Discussion

Development of a “molecular tracking device” enables tracking of antigen throughout the lymph node to specific cell types that acquire and archive antigens following subcutaneous immunization. Previous work used canonical surface markers to track antigen by microscopy and flow cytometry; instead, this approach simultaneously defines cell type by gene expression and quantifies the acquired antigen. The molecular tracking device enables the study of archived antigens at time points beyond the lifetime of antigen-fluorophore conjugates and provides a more complete catalog of cell types involved in antigen acquisition and retention.

This approach validates and expands upon previous work on antigen archiving and cell types that enhance protective immunity. Both here and in previous work, it was found that whereas LECs archive antigen, migratory DCs passing through the lymphatic vasculature are required to retrieve and present archived antigen to memory CD8 T cells derived from the initial infection or immunization s . Antigen exchange from LECs to DCs and subsequent DC presentation yields memory CD8 T cells with robust effector function during infectious challenge. Several recent reports defined LEC and non-endothelial stromal cell subsets within the lymph node^{51,52,53,54}. By combining the molecular tracking device described in this work with these reference cell types, it was found that non-endothelial stromal cell types acquire foreign antigens including CD34+ stromal cells, which neighbor subcapsular sinus LECs in the tissue⁵¹. These findings indicate that the interstitial pressure created by subcutaneous vaccination allows antigens to pass through the tissue directly to the LN capsule, bypassing the lymphatic capillaries. Intriguingly, bypass of lymphatic capillaries may still lead to LEC acquisition of antigens from the CD34+ stromal cells via SC-LEC exchange. Such a mechanism would encourage future LEC-DC interactions and provide a benefit to protective immunity.

Molecular tracking devices provide a measure of cell state orthogonal to gene expression, which was leveraged to identify candidate pathways involved in antigen acquisition (FIG. 4). It was shown that the caveolin pathway is upregulated in antigen-high LECs and confirmed this pathway is involved in antigen acquisition in vivo following vaccination via pharmacological inhibition of caveolar endocytosis (FIG. 4, panel f). Genes uniquely expressed by LECs such as Prox1, Cavin2 and Stab2^{50,79,81} represent targets for further manipulation of antigen archiving by LECs.

The psDNA component of the tracking device elicits an immune response similar to other TLR-antigen conjugate vaccines^{82,83}, likely due antigen-psDNA stability within dendritic cells that causes prolonged antigen presentation in the cells that acquire the antigen^{45,41}. This effect is illustrated by increased IFNγ production in the absence of ex vivo peptide stimulus (ova-psDNA compared to unconjugated ova; FIG. 2). Prolonged antigen presentation better replicates an infection wherein levels of viral or bacterial antigen rise over the duration of infection. However, in other applications, it can be useful to limit the immunoreactivity of the antigen-psDNA via cytosine methylation⁸⁴ or backbone modification⁸⁵.

Molecular tracking devices enable new approaches to study molecular dissemination in vivo. To date, protein-DNA conjugates have been deployed in single-cell mRNA sequencing experiments for ex vivo staining applications (e.g., CITE-seq⁴⁶). This work lays the groundwork for molecular tracking devices involving protein, antibody, drug, or pathogens conjugated to nuclease-resistant, barcoded oligonucleotides that are stable during transit through animal tissues. The approach naturally extends to understanding how multiple different antigens are processed (using unique DNA barcodes) and enables studies to manipulate antigen archiving to improve vaccines, vaccine formulations, and prime-boost strategies. Moreover, the oligonucleotide portion of the tracking device can enable analysis of its distribution in cells by in situ hybridization or intact tissue by spatial transcriptomics^{86,87,88}, obviating the need for antibody-mediated detection of antigen.

### REFERENCES

1. Manolova, V. et al. Nanoparticles target distinct dendritic cell populations according to their size. Eur J Immunol 38, 1404-1413 (2008).
2. Gerner, M. Y. et al. Dendritic cell and antigen dispersal landscapes regulate T cell immunity. J Exp Med 214, 3105-3122 (2017).
3. Bonneau, M. et al. Migratory monocytes and granulocytes are major lymphatic carriers of Salmonella from tissue to draining lymph node. J Leukoc Biol 79, 268-276 (2006).
4. Hampton, H. R. & Chtanova, T. Lymphatic Migration of Immune Cells. Front Immunol 10, 1168 (2019).
5. Eisenbarth, S. C. Dendritic cell subsets in T cell programming: location dictates function. Nat Rev Immunol 19, 89-103 (2019).
6. Jelley-Gibbs, D. M. et al. Unexpected prolonged presentation of influenza antigens promotes CD4 T cell memory generation. J Exp Med 202, 697-706 (2005).
7. Kim, T. S. et al. Antigen persistence and the control of local T cell memory by migrant respiratory dendritic cells after acute virus infection. J Exp Med 207, 1161-1172 (2010).
8. Kim, T. S. et al. T cell responses during influenza infection: getting and keeping control. Trends Immunol 32, 225-231 (2011).
9. Takamura, S. et al. The route of priming influences the ability of respiratory virus-specific memory CD8+ T cells to be activated by residual antigen. J Exp Med 207, 1153-1160 (2010).
10. Woodland, D. L. & Kohlmeier, J. E. Migration, maintenance and recall of memory T cells in peripheral tissues. Nat Rev Immunol 9, 153-161 (2009).
11. Zammit, D. J. et al. Residual antigen presentation after influenza virus infection affects CD8 T cell activation and migration. Immunity 24, 439-449 (2006).
12. Kedl, R. M. et al. Migratory dendritic cells acquire and present lymphatic endothelial cell-archived antigens during lymph node contraction. Nat Commun 8, 2034 (2017).
13. Kedl, R. M. & Tamburini, B. A. Antigen archiving by lymph node stroma: A novel function for the lymphatic endothelium. Eur J Immunol 45, 2721-2729 (2015).
14. Tamburini, B. A. et al. Antigen capture and archiving by lymphatic endothelial cells following vaccination or viral infection. Nat Commun 5, 3989 (2014).
15. Ahonen, C. L. et al. Combined TLR and CD40 triggering induces potent CD8+ T cell expansion with variable dependence on type I IFN. J Exp Med 199, 775-784 (2004).
16. Ahonen, C. L. et al. Enhanced efficacy and reduced toxicity of multifactorial adjuvants compared with unitary adjuvants as cancer vaccines. Blood 111, 3116-3125 (2008).
17. Badovinac, V.P. et al. Programmed contraction of CD8(+) T cells after infection. Nat Immunol 3, 619-626 (2002).
18. Corbin, G. A. & Harty, J. T. Duration of infection and antigen display have minimal influence on the kinetics of the CD4+ T cell response to Listeria monocytogenes infection. J Immunol 173, 5679-5687 (2004).
19. Kaech, S. M. & Ahmed, R. Memory CD8+ T cell differentiation: initial antigen encounter triggers a developmental program in naive cells. Nat Immunol 2, 415-422 (2001).
20. Kurche, J. S. et al. Comparison of OX40 ligand and CD70 in the promotion of CD4+ T cell responses. J Immunol 185, 2106-2115 (2010).
21. Kurche, J. S. et al. Type I IFN-dependent T cell activation is mediated by IFN-dependent dendritic cell OX40 ligand expression and is independent of T cell IFNR expression. J Immunol 188, 585-593 (2012).
22. Lucas, E. D. et al. Type 1 IFN and PD-L1 Coordinate Lymphatic Endothelial Cell Expansion and Contraction during an Inflammatory Immune Response. J Immunol 201, 1735-1747 (2018).
23. McWilliams, J. A. et al. Multiple innate signaling pathways cooperate with CD40 to induce potent, CD70-dependent cellular immunity. Vaccine 28, 1468-1476 (2010).
24. Sanchez, P. J. & Kedl, R. M. An alternative signal 3: CD8(+) T cell memory independent of IL-12 and type I IFN is dependent on CD27/OX40 signaling. Vaccine 30, 1154-1161 (2011).
25. Sanchez, P. J. et al. Combined TLR/CD40 stimulation mediates potent cellular immunity by regulating dendritic cell expression of CD70 in vivo. J Immunol 178, 1564-1572 (2007).
26. Tamburini, B. A. et al. IL-6-inducing whole yeast-based immunotherapy directly controls IL-12-dependent CD8 T-cell responses. J Immunother 35, 14-22 (2012).
27. Heesters, B. A. et al. Endocytosis and recycling of immune complexes by follicular dendritic cells enhances B cell antigen binding and activation. Immunity 38, 1164-1175 (2013).
28. Fletcher, A. L. et al. Lymph node fibroblastic reticular cells directly present peripheral tissue antigen under steady-state and inflammatory conditions. J Exp Med 207, 689-697 (2010).
29. Fletcher, A. L. et al. Lymph node stroma broaden the peripheral tolerance paradigm. Trends Immunol 32, 12-18 (2011).
30. Turley, S. J. et al. The stromal and haematopoietic antigen-presenting cells that reside in secondary lymphoid organs. Nat Rev Immunol 10, 813-825 (2010).
31. Cohen, J. N. et al. Lymph node-resident lymphatic endothelial cells mediate peripheral tolerance via Aire-independent direct antigen presentation. J Exp Med 207, 681-688 (2010).
32. Cohen, J. N. et al. Tolerogenic properties of lymphatic endothelial cells are controlled by the lymph node microenvironment. PLoS One 9, e87740 (2014).
33. Nichols, L. A. et al. Deletional self-tolerance to a melanocyte/melanoma antigen derived from tyrosinase is mediated by a radio-resistant cell in peripheral and mesenteric lymph nodes. J Immunol 179, 993-1003 (2007).
34. Rouhani, S. J. et al. Roles of lymphatic endothelial cells expressing peripheral tissue antigens in CD4 T-cell tolerance induction. Nat Commun 6, 6771 (2015).
35. Tewalt, E. F. et al. Lymphatic endothelial cells induce tolerance via PD-L1 and lack of costimulation leading to high-level PD-1 expression on CD8 T cells. Blood 120, 4772-4782 (2012).
36. Dubrot, J. et al. Lymph node stromal cells acquire peptide-MHCII complexes from dendritic cells and induce antigen-specific CD4(+) T cell tolerance. J Exp Med 211, 1153-1166 (2014).
37. Hirosue, S. et al. Steady-state antigen scavenging, cross-presentation, and CD8+ T cell priming: a new role for lymphatic endothelial cells. J Immunol 192, 5002-5011 (2014).
38. Lund, A. W. et al. VEGF-C promotes immune tolerance in B16 melanomas and cross-presentation of tumor antigen by lymph node lymphatics. Cell Rep 1, 191-199 (2012).
39. Jelley-Gibbs, D. M. et al. Persistent depots of influenza antigen fail to induce a cytotoxic CD8 T cell response. J Immunol 178, 7563-7570 (2007).
40. Stoeckius, M. et al. Simultaneous epitope and transcriptome measurement in single cells. Nat Methods 14, 865-868 (2017).
41. Xu, Z. & Moyle, P. M. Bioconjugation Approaches to Producing Subunit Vaccines Composed of Protein or Peptide Antigens and Covalently Attached Toll-Like Receptor Ligands. Bioconjug Chem 29, 572-586 (2018).
42. Baek, K. H. et al. A novel function of phosphorothioate oligodeoxynucleotides as chemoattractants for primary macrophages. J Immunol 167, 2847-2854 (2001).
43. Coffman, R. L. et al. Vaccine adjuvants: putting innate immunity to work. Immunity 33, 492-503 (2010).
44. Vollmer, J. et al. Oligodeoxynucleotides lacking CpG dinucleotides mediate Toll-like receptor 9 dependent T helper type 2 biased immune stimulation. Immunology 113, 212-223 (2004).
45. van Montfoort, N. et al. Antigen storage compartments in mature dendritic cells facilitate prolonged cytotoxic T lymphocyte cross-priming capacity. Proc Natl Acad Sci USA 106, 6730-6735 (2009).
46. Islam, S. et al. Quantitative single-cell RNA-seq with unique molecular identifiers. Nat Methods 11, 163-166 (2014).
47. Lucas, E. D. & Tamburini, B. A. J. Lymph Node Lymphatic Endothelial Cell Expansion and Contraction and the Programming of the Immune Response. Front Immunol 10, 36 (2019).
48. Fu, R. et al. clustifyr: an R package for automated single-cell RNA sequencing cluster classification. F1000Res 9, 223 (2020).
49. Brown, C. C. et al. Transcriptional Basis of Mouse and Human Dendritic Cell Heterogeneity. Cell 179, 846-863 e824 (2019).
50. Miller, J. C. et al. Deciphering the transcriptional network of the dendritic cell lineage. Nat Immunol 13, 888-899 (2012).
51. Rodda, L. B. et al. Single-Cell RNA Sequencing of Lymph Node Stromal Cells Reveals Niche-Associated Heterogeneity. Immunity 48, 1014-1028 e1016 (2018).
52. Fujimoto, N. et al. Single-cell mapping reveals new markers and functions of lymphatic endothelial cells in lymph nodes. PLoS Biol 18, e3000704 (2020).
53. Kalucka, J. et al. Single-Cell Transcriptome Atlas of Murine Endothelial Cells. Cell 180, 764-779 e720 (2020).
54. Xiang, M. et al. A Single-Cell Transcriptional Roadmap of the Mouse and Human Lymph Node Lymphatic Vasculature. Front Cardiovasc Med 7, 52 (2020).
55. Guilliams, M. et al. Dendritic cells, monocytes and macrophages: a unified nomenclature based on ontogeny. Nat Rev Immunol 14, 571-578 (2014).
56. Aliberti, J. et al. Essential role for ICSBP in the in vivo development of murine CD8alpha+dendritic cells. Blood 101, 305-310 (2003).
57. Hildner, K. et al. Batf3 deficiency reveals a critical role for CD8alpha+ dendritic cells in cytotoxic T cell immunity. Science 322, 1097-1100 (2008).
58. Tsujimura, H. et al. ICSBP/IRF-8 retrovirus transduction rescues dendritic cell development in vitro. Blood 101, 961-969 (2003).
59. Lewis, K. L. et al. Notch2 receptor signaling controls functional differentiation of dendritic cells in the spleen and intestine. Immunity 35, 780-791 (2011).
60. Schlitzer, A. et al. IRF4 transcription factor-dependent CD11b+ dendritic cells in human and mouse control mucosal IL-17 cytokine responses. Immunity 38, 970-983 (2013).
61. Bachem, A. et al. Expression of XCR1 Characterizes the Batf3-Dependent Lineage of Dendritic Cells Capable of Antigen Cross-Presentation. Front Immunol 3, 214 (2012).
62. Malhotra, D. et al. Transcriptional profiling of stroma from inflamed and resting lymph nodes defines immunological hallmarks. Nat Immunol 13, 499-510 (2012).
63. Breuilh, L. et al. Galectin-3 modulates immune and inflammatory responses during helminthic infection: impact of galectin-3 deficiency on the functions of dendritic cells. Infect Immun 75, 5148-5157 (2007).
64. Bune, A. J. et al. Mice lacking tartrate-resistant acid phosphatase (Acp 5) have disordered macrophage inflammatory responses and reduced clearance of the pathogen, Staphylococcus aureus. Immunology 102, 103-113 (2001).
65. Figueiredo, C. R. et al. Blockade of MIF-CD74 Signalling on Macrophages and Dendritic Cells Restores the Antitumour Immune Response Against Metastatic Melanoma. Front Immunol 9, 1132 (2018).
66. Gschwandtner, M. et al. More Than Just Attractive: How CCL2 Influences Myeloid Cell Behavior Beyond Chemotaxis. Front Immunol 10, 2759 (2019).
67. Hirano, M. et al. IgEb immune complexes activate macrophages through FcgammaRlV binding. Nat Immunol 8, 762-771 (2007).
68. Jin, X. et al. Pyruvate Kinase M2 Promotes the Activation of Dendritic Cells by Enhancing IL-12p35 Expression. Cell Rep 31, 107690 (2020).
69. Lammermann, T. & Kastenmuller, W. Concepts of GPCR-controlled navigation in the immune system. Immunol Rev 289, 205-231 (2019).
70. Mancardi, D. A. et al. FcgammaRlV is a mouse IgE receptor that resembles macrophage FcepsilonRI in humans and promotes IgE-induced lung inflammation. J Clin Invest 118, 3738-3750 (2008).
71. PrabhuDas, M. R. et al. A Consensus Definitive Classification of Scavenger Receptors and Their Roles in Health and Disease. J Immunol 198, 3775-3789 (2017).
72. Sinclair, N. R. Why so many coinhibitory receptors? Scand J Immunol 50, 10-13 (1999).
73. Crawford, A. et al. A role for the chemokine RANTES in regulating CD8 T cell responses during chronic viral infection. PLoS Pathog 7, e1002098 (2011).
74. Yamakita, Y. et al. Fascin1 promotes cell migration of mature dendritic cells. J Immunol 186, 2850-2859 (2011).
75. Harvey, N. L. et al. Lymphatic vascular defects promoted by Proxl haploinsufficiency cause adult-onset obesity. Nat Genet 37, 1072-1081 (2005).
76. Hong, Y. K. et al. Proxl is a master control gene in the program specifying lymphatic endothelial cell fate. Dev Dyn 225, 351-357 (2002).
77. Wigle, J. T. & Oliver, G. Proxl function is required for the development of the murine lymphatic system. Cell 98, 769-778 (1999).
78. Triacca, V. et al. Transcellular Pathways in Lymphatic Endothelial Cells Regulate Changes in Solute Transport by Fluid Stress. Circ Res 120, 1440-1452 (2017).
79. Malhotra, D. et al. Transcriptional profiling of stroma from inflamed and resting lymph nodes defines immunological hallmarks. Nat Immunol 13, 499-510 (2012).
80. Miller, C. M. et al. Stabilin-1 and Stabilin-2 are specific receptors for the cellular internalization of phosphorothioate-modified antisense oligonucleotides (ASOs) in the liver. Nucleic Acids Res 44, 2782-2794 (2016).
81. Heng, T. S., Painter, M. W. & Immunological Genome Project, C. The Immunological Genome Project: networks of gene expression in immune cells. Nat Immunol 9, 1091-1094 (2008).
82. Oh, J. Z. & Kedl, R. M. The capacity to induce cross-presentation dictates the success of a TLR7 agonist-conjugate vaccine for eliciting cellular immunity. J Immunol 185, 4602-4608 (2010).
83. Oh, J. Z. et al. TLR7 enables cross-presentation by multiple dendritic cell subsets through a type I IFN-dependent pathway. Blood 118, 3028-3038 (2012).
84. Hemmi, H. et al. A Toll-like receptor recognizes bacterial DNA. Nature 408, 740-745 (2000).
85. Lange, M. J. et al. Activation of Innate Immune Responses by a CpG Oligonucleotide Sequence Composed Entirely of Threose Nucleic Acid. Nucleic Acid Ther 29, 51-59 (2019).
86. Eng, C. L. et al. Transcriptome-scale super-resolved imaging in tissues by RNA seqFISH. Nature 568, 235-239 (2019).
87. Rodrigues, S. G. et al. Slide-seq: A scalable technology for measuring genome-wide expression at high spatial resolution. Science 363, 1463-1467 (2019).
88. Stahl, P. L. et al. Visualization and analysis of gene expression in tissue sections by spatial transcriptomics. Science 353, 78-82 (2016).
89. van Buggenum, J. A. et al. A covalent and cleavable antibody-DNA conjugation strategy for sensitive protein detection via immuno-PCR. Sci Rep 6, 22675 (2016).
90. Anis, M. M. et al. Modulation of naive CD4+ T-cell responses to an airway antigen during pulmonary mycobacterial infection. Infect Immun 75, 2260-2268 (2007).
91. Roederer, M. Interpretation of cellular proliferation data: avoid the panglossian. Cytometry. Part A: the journal of the International Society for Analytical Cytology 79, 95-101 (2011).

Whereas specific embodiments of the present inventive concept have been shown and described, it will be understood that other modifications, substitutions, and alternatives are apparent to one of ordinary skill in the art. Such modifications, substitutions and alternatives can be made without departing from the spirit and scope of the inventive concept, which should be determined from the appended claims.

1. A molecular conjugate comprising:
a molecule or molecular structure of interest; and
a nucleic acid linked to the molecule or molecular structure of interest,
wherein the nucleic acid comprises a nucleic acid amplification primer binding sequence, a barcode identifier sequence, and a capture sequence, and wherein the nucleic acid comprises a phophorothioate modification at every nucleotide linkage.

2. The molecular conjugate of claim 1, wherein the nucleic acid comprises DNA.

3. The molecular conjugate of claim 1, wherein the nucleic acid is functionalized with trans-cyclooctene, the molecule or molecular structure of interest is functionalized with methyltetrazine, and wherein the nucleic acid and antigen are conjugated together through an inverse electron demand Diels-Alder (IEDDA) reaction.

4. The molecular conjugate of claim 3, wherein the nucleic acid is functionalized with trans-cyclooctene through a 5′ amine modification of the nucleic acid.

5. The molecular conjugate of claim 4, wherein the nucleic acid further comprises a 3′ biotin modification of the nucleic acid.

6. The molecular conjugate of claim 1, wherein the molecule or molecular structure is an antigen or is antigenic.

7. The molecular conjugate of claim 1, wherein the molecular conjugate is nonimmunogenic.

8. The molecular conjugate of claim 1, wherein the molecule or molecular structure is a component in and/or an ingredient in a vaccine.

9. The molecular conjugate of claim 1, wherein the molecule or molecular structure of interest comprises a peptide or protein.

10. The molecular conjugate of claim 9, wherein the peptide or protein is a viral peptide or protein.

11. The molecular conjugate of claim 10, wherein the viral peptide or protein comprises an influenza peptide or protein.

12. The molecular conjugate of claim 10, wherein the viral peptide or protein comprises a coronavirus peptide or protein.

13. The molecular conjugate of claim 12, wherein the coronavirus peptide or protein is a SARS-CoV2 peptide or protein.

14. The molecular conjugate of claim 9, wherein the peptide or protein is derived from ovalbumin.

15. A composition or formulation comprising the molecular conjugate of claim 1, and a pharmaceutically acceptable carrier.

16. The composition or formulation of claim 15, wherein the composition or formulation is suitable for parenteral administration.

17. The composition or formulation of claim 16, wherein the parenteral administration is intravenous, intraperitoneal, subcutaneous, or intradermal administration.

18. The composition or formulation of claim 15, wherein the composition or formulation is suitable for nasal, intranasal, or inhalational administration.

19. The composition or formulation of claim 15, wherein the composition or formulation is suitable for oral administration.

20. A kit comprising the molecular conjugate, composition, or formulation of claim 1, at least one oligonucleotide amplification primer, and instructions for use of the kit.

21. A method of tracking acquisition of a molecule or molecular structure of interest in cells comprising:
performing single-cell nucleic acid sequencing on cells of interest isolated from a subject exposed to the molecular conjugate, composition, or formulation of claim 1; and
analyzing data acquired from the single-cell nucleic acid sequencing for a presence of the barcode identifier sequence,
wherein the presence of the barcode identifier sequence is indicative of acquisition of the molecule or molecular structure of interest by a particular cell or cells.

22. The method of claim 21, wherein the single-cell nucleic acid sequencing comprises capturing nucleic acids and a nucleic acid amplification step.

23. The method of claim 21, wherein the single-cell nucleic acid sequencing comprises single-cell mRNA sequencing.

24. The method of claim 21, wherein the particular cell or cells comprise a cell or cells isolated from an organ of the subject.

25. The method of claim 21, wherein the particular cell or cells comprise a cell or cells from a tissue sample or a tissue slice.

26. The method of claim 21, wherein the particular cell or cells comprise cells from a lymphatic organ, lymphatic tissue, and/or lymph node.

27. The method of claim 21, wherein the particular cell or cells comprise stromal cells (SCs).

28. The method of claim 27, wherein the SCs comprise follicular dendritic cells (FDCs) and/or lymphatic endothelial cells (LECs).

29. The method of claim 21, wherein molecule or molecular structure acquisition is tracked at a plurality of time points and/or over a period of time.

30. The method of claim 21, wherein the subject is a human subject.

31. A method of tracking distribution or location of a molecule or a molecular structure of interest in a subject comprising:
performing single-cell nucleic acid sequencing on cells of interest isolated from a subject exposed to the molecular conjugate, composition, or formulation of claim 1; and
analyzing data acquired from the single-cell nucleic acid sequencing for a presence of the barcode identifier sequence,
wherein the presence of the barcode identifier sequence is indicative of acquisition of the molecule or molecular structure of interest by a particular cell or cells in the subject, and where the molecule or molecular structure of interest is distributed or located in the subject.

32. The method of claim 31, wherein the single-cell nucleic acid sequencing comprises capturing nucleic acids and a nucleic acid amplification step.

33. The method of claim 31, wherein the single-cell nucleic acid sequencing comprises single-cell mRNA sequencing.

34. The method of claim 31, wherein the particular cell or cells comprise a cell or cells isolated from an organ of the subject.

35. The method of claim 31, wherein the particular cell or cells comprise a cell or cells from a tissue sample or a tissue slice.

36. The method of claim 31, wherein the particular cell or cells comprise cells from a lymphatic organ, lymphatic tissue, and/or lymph node.

37. The method of claim 31, wherein the particular cell or cells comprise SCs.

38. The method of claim 37, wherein the SCs comprise FDCs and/or LECs.

39. The method of claim 31, wherein molecule or molecular structure acquisition is tracked at a plurality of time points and/or over a period of time.

40. The method of claim 31, wherein the subject is a human subject.

41. A method of molecular tracking comprising:
performing single-cell nucleic acid sequencing on cells of interest isolated from a subject exposed to the molecular conjugate, composition, or formulation of claim 1; and
analyzing data acquired from the single-cell nucleic acid sequencing for a presence of the barcode identifier sequence,
wherein the presence of the barcode identifier sequence is indicative of acquisition of the molecule of interest by a particular cell or cells.

42. The method of claim 41, wherein the single-cell nucleic acid sequencing comprises capturing nucleic acids and a nucleic acid amplification step.

43. The method of claim 41, wherein the single cell nucleic acid sequencing comprises single-cell mRNA sequencing.

44. The method of claim 41, wherein the particular cell or cells comprise a cell or cells isolated from an organ of the subject.

45. The method of claim 41, wherein the particular cell or cells comprise a cell or cells from a tissue sample or a tissue slice.

46. The method of claim 41, wherein the particular cell or cells comprise cells from a lymphatic organ, lymphatic tissue, and/or lymph node.

47. The method of claim 41, wherein the particular cell or cells comprise SCs.

48. The method of claim 47, wherein the SCs comprise FDCs and/or LECs.

49. The method of claim 41, wherein the molecular tracking is performed at a plurality of time points and/or over a period of time.

50. The method of claim 41, wherein the subject is a human subject.

51. A method of tracking antigen archiving comprising:
performing single-cell nucleic acid sequencing on cells of interest isolated from a subject exposed to a molecular conjugate comprising an antigen of interest linked to a nucleic acid, wherein the nucleic acid comprises a nucleic acid amplification primer binding sequence, a barcode identifier sequence, and a capture sequence, and wherein the nucleic acid comprises a phophorothioate modification at every nucleotide linkage; and
analyzing data acquired from the single-cell nucleic acid sequencing for a presence of the barcode identifier sequence,
wherein the presence of the barcode identifier sequence is indicative of acquisition of the antigen of interest by a particular cell or cells.

52. The method of claim 51, wherein the single-cell nucleic acid sequencing comprises capturing nucleic acids and a nucleic acid amplification step.

53. The method of claim 51, wherein the single cell nucleic acid sequencing comprises single-cell mRNA sequencing.

54. The method of claim 51, wherein the antigen comprises a peptide or protein.

55. The method of claim 54, wherein the peptide or protein comprises an influenza peptide or protein.

56. The method of claim 54, wherein the peptide or protein comprises a coronavirus peptide or protein.

57. The method of claim 56, wherein the coronavirus peptide or protein is a SARS-CoV2 peptide or protein.

58. The method of claim 54, wherein the peptide or protein is derived from ovalbumin.

59. The method of claim 51, wherein the particular cell or cells comprise a cell or cells isolated from an organ of the subject.

60. The method of claim 51, wherein the particular cell or cells comprise a cell or cells from a tissue sample or a tissue slice.

61. The method of claim 51, wherein the particular cell or cells comprise cells from a lymphatic organ, lymphatic tissue, and/or lymph node.

62. The method of claim 51, wherein the particular cell or cells comprise SCs.

63. The method of claim 62, wherein the SCs comprise FDCs and/or LECs.

64. The method of claim 51, wherein antigen archiving is tracked at a plurality of time points and/or over a period of time.

65. The method of claim 51, wherein the subject is a human subject.
